(19) United States
(12) Patent Application Publication
(10) Pub. No.: US 2026/0098308 A1
(43) Pub. Date: Apr. 9, 2026

(54) Gene Transcripts as Signatures for Tead-Active Cancer

(71) Applicant: Sanofi, Paris (FR)

(72) Inventors: Manoel Nunes, Paris (FR); Emmanuel Spanakis, Paris (FR)

(21) Appl. No.: 19/469,778

(22) PCT Filed: Mar. 29, 2024
(86) PCT No.: PCT/EP2024/058750

§ 371 Date: Sep. 26, 2025

Related U.S. Application Data
(60) Provisional application No. 63/456,386, filed on Mar. 31, 2023.
(60) Provisional application No. 63/459,501, filed on Apr. 14, 2023.

(30) Foreign Application Priority Data
May 23, 2023 (EP) 23305810.6

(51) Int. Cl. C12Q 1/6886

(52) U.S. Cl. CPC C12Q 1/6886; C12Q 2600/106; C12Q 2600/136; C12Q 2600/158

(57) ABSTRACT

The invention relates to an isolated set of gene transcripts (A) from a set of genes consisting of a subset of genes (1) consisting of ADM, AXL, BIRC5, CDV3, CRIM1, CTGF, CYR61, FSTL1, GADD45A, KRT8, LMNB2, MATN2, PKP4, RND3, RPS24, SEC14L1, SGK1, SLC25A3, SLC3A2, TNFRSF12A, TPM1, TPX2, TUBB6 and of a subset of genes (2) consisting of CTSB, FTH1, SQSTM1, TCF25, UBC, or an isolated set of genes (B) consisting of at least one gene transcript from a set of genes consisting of DLC1, AKAP2, CANX, SAFB2, EIF4H, NDUFS5, SEPT9, and EIF4A1, and their use in diagnostic methods for TEAD-active cancer.

### TECHNICAL FIELD

The disclosure relates to the field of gene transcripts as signatures of TEAD-active cancer.

The disclosure relates to sets of gene transcripts, and the measure of their levels for uses and methods for characterizing cancer, for monitoring cancer evolution and cancer response to TEAD-inhibitor treatment, as well as tools for screening TEAD-inhibitor candidate compounds.

### TECHNICAL BACKGROUND

In normal tissues, the transcription co-factors YAP1 and WWTR1 bind to transcription factors of the TEAD family (TEAD1, TEAD2, TEAD3, and TEAD4), collectively referred to as “TEAD,” to form an active protein complex. This complex recognizes and binds to a specific sequence motif in the promoters of target genes and initiates or inhibits their transcription. Cell proliferation, survival, plasticity, and migration necessary for normal biological processes such as organ growth and wound healing are therefore regulated by TEAD (Totaro A, et al. Nat Cell Biol. 2018; 20(8):888-899. doi: 10.1038/s41556-018-0142-z). In intact adult mammalian tissues, YAP1 and WWTR1 are generally phosphorylated by kinases of the Hippo pathway, and thereby retained in the cytoplasm and degraded. In this case, there is no active transcription complex in the nucleus (Totaro A, et al. Nat Cell Biol 2018. August; 20(8):888-899).

Over the past few years, YAP1, WWTR1, their TEAD partners, and their upstream regulators—notably the tumor suppressor pathway known as Hippo pathway—present increasing interest in cancer research (Zhang N et al. Dev Cell. 2010 Jul. 20; 19(1):27-38. doi: 10.1016/j.devcel.2010.06.015; Lu L, et al. Proc Natl Acad Sci USA. 2010; 107(4):1437-42. doi: 10.1073/pnas.0911427107; Nishio M, et al. Proc Natl Acad Sci USA. 2016; 113(1):E71-80. doi: 10.1073/pnas.1517188113; Liu-Chittenden Y, et al., Genes Dev. 2012; 26(12):1300-5. doi: 10.1101/gad.192856.112). Mutations or physiological dysregulation of either YAP1, WWTR1, or any of their upstream regulators, may result in insufficient phosphorylation and degradation of these proteins. Without phosphorylation and degradation, the transcription co-factors enter the nucleus, bind to TEAD, and initiate oncogenic transcription. Increased activity of either component of the complex may increase, or decrease, the transcription of downstream effectors (TEAD-dependent transcription). Some of the modulated genes are direct targets of TEAD bearing TEAD-binding motifs (Zanconato, F. et al. Nat Cell Biol 2015:17:1218-1227). Others are modulated indirectly. Thus, the Hippo-YAP1/WWTR1-TEAD pathway (hereinafter “TEAD pathway” or “TEAD signalling”) may directly trigger tumorigenesis or render existing tumors resistant to targeted treatments (Reggiani Fet al. Biochim Biophys Acta Rev Cancer. 2020:1873(1):188341. doi: 10.1016/j.bbcan.2020.188341; Kurppa K J et al. Cancer Cell. 2020; 37(1):104-122.e12. doi: 10.1016/j.ccell.2019.12.006).

For example, mutations and deletions of Hippo genes such as LATS2 and NF2 account for a large proportion of malignant mesothelioma tumors (Sekido Y et al. Cancers (Basel). 2018; 10(4):90. doi: 10.3390/cancers10040090). YAP1 is found focally amplified, and over-expressed at both RNA and protein levels, in several types of tumors, notably in cervical cancers (Zanconato F et al. Cancer Cell. 2016; 29(6):783-803. doi: 10.1016/j.ccell.2016.05.005). Activation of TEAD-dependent transcription may give tumor cells a mesenchymal stem cell-like phenotype since notorious stem-cell transcription factors like SOX2, OCT3/4, NANOG and MYC are also controlled by TEAD (Bora-Singhal N et al. Stem Cells. 2015; 33(6):1705-18. doi: 10.1002/stem.1993). Cancers for which tumor cells have a TEAD-active pathway are named TEAD-active cancer.

To measure the transcriptional activity of such complexes, estimate the proportion of cases where TEAD-dependent transcription may be responsible for tumor development or evolution, and evaluate the target engagement and pharmacodynamics of novel TEAD-pathway inhibitors, sensitive and easy to implement gene transcripts signature (or transcriptional signatures) are needed.

The field of liquid biopsies is of enormous interest for non-invasive monitoring of cancer progression and response to treatment. Extracellular Vesicles (EVs) such as exosomes, micro-vesicles (ectosomes), apoptotic bodies, exosomes-like, and others, are nanoparticles found in all biological fluids (body fluids). These cell-derived, small, secreted vesicles convey biological information, either by surface-to-surface interaction or by shuttling bioactive molecules to a recipient cell's cytoplasm. Because EVs harbor or encapsulate the cargos of their parent cells, their contents may be useful as biomarkers to follow drug activity (Graea Raposo and Willem Stoorvogel. 2013. Extracellular vesicles: exosomes, microvesicles, and friends. J. Cell Biol.: 200 (4): 373-83; Nunes et al.; 2020.Tumor-derived Extracellular Vesicles (EVs) expressing TGFβ as potential biomarkers for anti-TGFβ antibody activity and drug activity in liquid biopsy. AACR2020; Poster #773; Urabe et al.; 2020. Extracellular vesicles as biomarkers and therapeutic targets for cancer. Am. J. Physiol Cell Physiol.:318 (1); Calvet, L., Dos-Santos, O., Spanakis, E. et al. 2022. YAP1 is essential for malignant mesothelioma tumor maintenance. BMC Cancer 22, 639. doi.org/10.1186/s12885-022-09686).

Therefore, there is a need for new set of gene transcripts usable as signatures for diagnosing or monitoring TEAD-active cancer.

There is a need for cost-effective diagnostic tools and methods for diagnosing or monitoring TEAD-active cancer.

There is a need for new gene transcripts usable as signatures for TEAD-active cancer which can be simple, reliable and robust to use.

There is a need for improved sets of gene transcripts which can be used in robust and reliable methods for characterizing cancer as TEAD-active, for monitoring or predicting the evolution of a cancer or the response of a cancer to a TEAD-inhibitor treatment, or for screening new TEAD-inhibitor candidate compounds.

There is a need for new sets of gene transcripts which can be used as signature of TEAD-active cancer by measure of their levels.

There is a need for an improved non-invasive method for obtaining a gene transcripts signature for characterizing or diagnosing or monitoring a TEAD-active cancer.

There is a need for new simple and reliable methods for obtaining gene transcripts signature of TEAD-active cancer.

There is a need for new sets of gene transcripts as signature of TEAD-active cancer which can be obtained from extracellular vesicles.

The present disclosure has for purpose to satisfy all or parts of those needs.

### SUMMARY

According to one of its objects, the present disclosure relates to an isolated set of gene transcripts, designated thereafter as set of gene transcripts (A), from a set of genes consisting of a subset of genes (1) consisting of ADM, AXL, BIRC5, CDV3, CRIM1, CTGF, CYR61, FSTL1, GADD45A, KRT8, LMNB2, MATN2, PKP4, RND3, RPS24, SEC14L1, SGK1, SLC25A3, SLC3A2, TNFRSF12A, TPM1, TPX2, and TUBB6 and of a subset of genes (2) consisting of CTSB, FTH1, SQSTM1, TCF25, and UBC. According to the context, (A) may refer to the set of genes or to its corresponding set of gene transcripts.

An isolated set of gene transcripts (A) of the disclosure is obtained from a set of genes, the set of genes consists of a first subset of genes (1) and of a second subset of genes (2), the first subset of genes (1) consisting of ADM, AXL, BIRC5, CDV3, CRIM1, CTGF, CYR61, FSTL1, GADD45A, KRT8, LMNB2, MATN2, PKP4, RND3, RPS24, SEC14L1, SGK1, SLC25A3, SLC3A2, TNFRSF12A, TPM1, TPX2, and TUBB6 and the second subset of genes (2) consisting of CTSB, FTH1, SQSTM1, TCF25, and UBC.

According to one of its objects, the present disclosure relates to an isolated set of gene transcripts, designated thereafter as set of gene transcripts (B), consisting of at least one gene transcript from a set of genes consisting of DLC1, AKAP2, CANX, SAFB2, EIF4H, NDUFS5, SEPT9, and EIF4A1. According to the context, (B) may refer to the set of genes or to its corresponding set of gene transcripts.

According to one of its objects, the present disclosure relates to an isolated set of gene transcripts, designated thereafter as set of gene transcripts (B), consisting of at least two gene transcripts, one being a gene transcript from gene DLC1 and the at least second gene transcript being from a set of genes consisting of AKAP2, CANX, SAFB2, EIF4H, NDUFS5, SEPT9, and EIF4A1.

As shown in the Example section, the newly proposed sets of genes transcripts (A) or (B) can be used as reliable and sensitive signature for characterizing and monitoring TEAD-active cancer. Further, as shown in the Example section, the gene transcripts can be easily isolated from Extracellular Vesicles (EVs). The measure of their levels provides a transcriptional signature allowing identification of TEAD-active cancer and allowing monitoring changes in TEAD activity so as to monitor or predict evolution of a cancer or a response of cancer to a treatment or to screen new TEAD-inhibitor treatment.

In the Examples, the utility of EVs-encapsulated and harbored mRNAs has been assessed as a transcriptional signature of the TEAD pathway activity and it was shown that the EVs can be used as a source of gene transcripts for the measure of a signature of TEAD-active cancer. EVs can be isolated from various body fluid samples, such as urine, saliva, or blood samples, allowing to implement non-invasive methods, e.g., by obtaining saliva, blood, or urine samples, for measuring a transcriptional signature for TEAD-active cancers.

Further, the Examples show that the newly disclosed sets of gene transcripts isolated from EVs can be used to monitor the inhibition of YAP1/TEAD signaling pathway by a TEAD-inhibitor (TEADi).

The Examples further show that the newly identified sets of gene transcripts obtained from EVs shed by tumor cell lines were able to be used to compute TEAD-activity scores, and that the calculated scores correlated with the TEAD-activity score using the TEAD-500 signature previously described in PCT/EP2023/057332 or as YAP1-TEAD activity signature in Calvet et al. (BMC Cancer, 2022, 22:639, doi.org/10.1186/s12885-022-09686-y) and calculated from parent-cell mRNA. The results show that the TEAD activity of parent tumor cells can be measured with confidence in the secreted EVs using the newly identified sets of gene transcripts and their levels. Therefore, sets of gene transcripts obtained from EVs provide relevant pharmacodynamic (PD) markers to monitor TEAD activity and TEAD inhibition by TEAD-inhibitor treatment.

The newly identified sets of gene transcripts obtained from extracellular vesicles, and their levels, can be advantageously translated into a non-invasive, liquid biopsy-based transcriptional signature and biomarker for clinical use.

The identified sets of gene transcripts may be utilized in a variety of clinical and research-related utilities, including, but not limited to, monitoring the activity of the TEAD complex and evaluating the pharmacodynamics of novel inhibitors of the TEAD pathway in vitro or in vivo, to diagnose tumors of which the development or evolution is attributable to activation of the TEAD complex, to recruit patients with active TEAD to clinical trials designed to evaluate TEAD pathway inhibitors, to prognose survival, response to, and benefit from an anti-TEAD pathway treatment, alone or in combination with other treatments, to estimate the proportion of cases within cohorts, for each cancer indication or subtype, where TEAD-dependent transcription may be responsible for tumor development or evolution to treatment resistance, and to measure TEAD-dependent transcription in any tissue, under any condition, pathological or not, in the laboratory or in the clinic.

In some embodiments, a set of gene transcripts (B) may consist in at least 2, 3, 4, 5, 6, 7, or consisting of 8 gene transcripts from a set of genes consisting of DLC1, AKAP2, CANX, SAFB2, EIF4H, NDUFS5, SEPT9, and EIF4A1.

In some embodiments, a set of gene transcripts (B) may consist in a set of genes consisting of at least two gene transcripts, one being a gene transcript from gene DLC1 and the at least second gene transcript being from a set of genes consisting of AKAP2, CANX, SAFB2, EIF4H, NDUFS5, SEPT9, and EIF4A1.

In some embodiments, a set of gene transcripts of the disclosure may be obtained from isolated extracellular vesicles.

According to another of its objects, the disclosure relates to isolated extracellular vesicles comprising a set of gene transcripts as disclosed herein.

In some embodiments, the disclosure relates to isolated extracellular vesicles comprising a set of gene transcripts (A) obtained from a set of genes, the set of genes consists of a first subset of genes (1) and of a second subset of genes (2), the first subset of genes (1) consisting of ADM, AXL, BIRC5, CDV3, CRIM1, CTGF, CYR61, FSTL1, GADD45A, KRT8, LMNB2, MATN2, PKP4, RND3, RPS24, SEC14L1, SGK1, SLC25A3, SLC3A2, TNFRSF12A, TPM1, TPX2, and TUBB6 and the second subset of genes (2) consisting of CTSB, FTH1, SQSTM1, TCF25, and UBC.

In some embodiments, the disclosure relates to isolated extracellular vesicles comprising a set of gene transcripts consisting of at least one gene transcript from a set of genes consisting of DLC1, AKAP2, CANX, SAFB2, EIF4H, NDUFS5, SEPT9, and EIF4A1.

In some embodiments, a set of gene transcripts according to the disclosure may be for use as a biomarker of a TEAD activity, for measuring or characterizing a TEAD activity of a cancer or of a cell culture, or for use in a TEAD-inhibitor candidate compound screening method, or for use in a cancer diagnostic method.

A cancer diagnostic method may be selected among a method for characterizing a TEAD activity status of a cancer, a method for measuring a TEAD activity in a biological sample, a method for predicting a response of a cancer to a TEAD-inhibitor treatment, a method for monitoring a response of a cancer to a TEAD-inhibitor treatment, a method for predicting a TEAD-active cancer progression or regression, and a method for monitoring a TEAD-active cancer progression or regression.

According to another of its objects, the disclosure relates to a use of a set of gene transcripts disclosed herein, for measuring a TEAD activity in a biological sample.

According to another of its objects, the disclosure relates to a use of a set of gene transcripts disclosed herein, for characterizing a TEAD activity in a biological sample.

According to another of its objects, the disclosure relates to a use of a set of gene transcripts disclosed herein, for measuring a TEAD activity of a cancer from a subject in need thereof.

According to another of its objects, the disclosure relates to a use of a set of gene transcripts disclosed herein, for characterizing a TEAD activity status of a cancer in a subject in need thereof.

According to another of its objects, the disclosure relates to a use of a set of gene transcripts disclosed herein, for predicting a response of a cancer to a TEAD-inhibitor treatment in a subject in need thereof. The subject may be known or presumed to have a TEAD-active cancer.

According to another of its objects, the disclosure relates to a use of a set of gene transcripts disclosed herein, for monitoring a response of a cancer to a TEAD-inhibitor treatment in a subject in need thereof. The subject may be known or presumed to have a TEAD-active cancer.

According to another of its objects, the disclosure relates to a use of a set of gene transcripts disclosed herein, for predicting a cancer progression or regression in a subject in need thereof. The subject may be known or presumed to have a TEAD-active cancer.

According to another of its objects, the disclosure relates to a use of a set of gene transcripts disclosed herein, for monitoring a cancer progression or regression in a subject in need thereof. The subject may be known or presumed to have a TEAD-active cancer.

According to another of its objects, the disclosure relates to a use of a set of gene transcripts disclosed herein, for screening a TEAD-inhibitor candidate compound.

In some embodiments, the set of gene transcripts may be obtained from isolated extracellular vesicles.

According to another of its objects, the disclosure relates to a use of isolated extracellular vesicles for the above indicated uses.

In some embodiments, a level of each gene transcript may be obtained.

In some embodiments, a transcriptional signature may be obtained from the gene transcript levels.

In some embodiments, the obtained transcriptional signature may be compared to a transcriptional signature of reference.

In some embodiments, an observed deviation between the obtained transcriptional signature and the transcriptional signature of reference may be indicative of a cancer being TEAD-active or TEAD-inactive, or of a cancer susceptible to be responsive or not to the TEAD-inhibitor treatment, or of an effective or ineffective TEAD-inhibitor treatment, or of a TEAD-active cancer susceptible to progress or regress, or of a progressing or regressing TEAD-active cancer, or of a TEAD-inhibitor candidate compound being effective or ineffective.

In some embodiments, the gene transcript level may be subject to a mathematical normalization.

In some embodiments, when a set of gene transcripts (A) is used, then the mathematical normalization may compute a deR score according to a method comprising the steps of:
a) for each gene transcript of said set of genes, converting the level of each gene transcript into a fractional rank by dividing the rank of said gene having said level of said gene transcript by the number of genes from said set of genes,
b) isolating from the fractional ranks obtained at step a) the fractional rank obtained for the genes of the subset of genes (1) and computing their mean fractional rank (MFR-subset(1) or MFR-positive),
c) isolating from the fractional ranks obtained at step a) the fractional rank obtained for the genes of the subset of genes (2) and computing their mean fractional rank (MFR-subset(2) or MFR-negative), and
d) computing the deR score as MFR-subset(1) less MFR-subset(2).

In some embodiments, when a set of gene transcripts consisting of at least one gene transcript from a set of genes consisting of DLC1, AKAP2, CANX, SAFB2, EIF4H, NDUFS5, SEPT9, and EIF4A1 (set of gene transcripts from a set of genes (B)) is used, then the mathematical normalization may compute a (S) score according to a method comprising the steps of:
a) multiplying each level of each gene transcript of said set of genes by a coefficient associated with each gene, to obtain, for each gene, a product (Pgeneᵢ) wherein gene, refers to a gene listed in said set of genes;
b) summing the products Pgeneᵢ (ΣPgeneᵢ) obtained at step a) and adding a constant for obtaining a (S) score: (ΣPgeneᵢ+constant),
wherein the coefficient(s) and the constant used at steps a) and b) may be previously obtained by a stepwise multiple linear regression analysis correlating (i) levels of said gene transcripts previously obtained in a first biological sample with (ii) levels of gene transcripts of a TEAD-500 signature previously measured in a second biological sample, wherein the TEAD-500 signature comprises the gene transcripts of a set of genes comprising any of 220 to 249 of genes of a subset of genes (1) and any of 210 to 233 of genes of a subset of genes (2) as disclosed in Table 2. The first and second biological samples are representative of a same TEAD-active cancer. The first and second biological samples are representative of the cancer for which a use according to the disclosure is considered. The first sample may be a body fluid sample or a fraction thereof. The second sample may be a cancer cell sample.

In some embodiments, in the methods and uses of the disclosure, the correlation operated with the stepwise multiple linear regression analysis may be carried out between (i) the levels of said gene transcripts and (ii) a TEAD-score obtained with the levels of gene transcripts of a TEAD-500 signature.

In some embodiments, the computed deR or (S) score may be compared to a value of reference.

In some embodiments, an observed deviation between the computed deR or (S) score and the value of reference may be indicative of a cancer being TEAD-active or TEAD-inactive, or of a cancer susceptible to be responsive or not to the TEAD-inhibitor treatment, or of an effective or ineffective TEAD-inhibitor treatment, or of a TEAD-active cancer susceptible to progress or regress, or of a progressing or regressing TEAD-active cancer, or of a TEAD-inhibitor candidate compound being effective or ineffective.

In some embodiments, the reference value may be a first deR or (S) score and the deR or (S) score to be compared to said value of reference may be a second deR or (S) score subsequently measured to the first deR or (S) score.

In some embodiments, the level of each gene transcript may be obtained by RNA sequencing (RNA-seq) and may be quantified as FPKM (Fragments Per Kilobase of transcript per Million mapped reads).

According to another of its objects, the disclosure relates to a method for characterizing a TEAD activity status of a cancer in a subject in need thereof.

In some embodiments, the method may comprise the use of a set of gene transcripts from a set of genes (A) as disclosed herein, and then the method comprises at least the steps of:
a) obtaining, from a biological sample obtained from said subject, a level of each gene transcript of said set of genes,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a deR score as MFR-subset(1) less MFR-subset(2),
In some embodiments, the method may comprise the use of a set of gene transcripts from a set of genes (B) as disclosed herein, and then the method comprises at least the steps of:
a) obtaining, from a biological sample obtained from said subject, a level of each gene transcript of said set of genes,
b) multiplying each obtained level of each gene transcript of said set of genes obtained at step a) by a coefficient associated with each gene, to obtain, for each gene, a product (Pgeneᵢ) wherein gene; refers to a gene listed in said set of genes;
c) summing the products Pgeneᵢ (ΣPgeneᵢ) obtained at step b) and adding a constant for obtaining a (S) score: (ΣPgeneᵢ+constant),
wherein the coefficient(s) and the constant used at steps b) and c) are previously obtained by a stepwise multiple linear regression analysis correlating (i) levels of said gene transcripts previously obtained in a first biological sample obtained from a subject having said cancer with (ii) levels of gene transcripts of a TEAD-500 signature measured in a second biological sample from said subject, the second biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed, thereafter, in Table 2. The subject from whom are taken the first and second biological samples may be the same or may be different from the subject from whom is taken a biological sample for computing a (S) score. The first and second biological samples are representative of the cancer for which the method according to the disclosure is considered.

In some embodiments, in the methods of the disclosure, the correlation operated with the stepwise multiple linear regression analysis may be carried between (i) the levels of said gene transcripts and (ii) a TEAD-score obtained with the levels of gene transcripts of a TEAD-500 signature.

According to another of its objects, the disclosure relates to a method for monitoring a progression or a regression of a cancer in a subject presumed or known to have a TEAD-active cancer, the method comprising the use of a set of gene transcripts from a set of genes (A), and the method comprising at least the steps of:
a) obtaining, from a first biological sample obtained from said subject at a first time, a level of each gene transcript of said set of genes,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a first deR score as MFR-subset(1) less MFR-subset(2),
f) obtaining, from a second biological sample obtained from said subject at a second time, subsequent to the first time, a level of each gene transcript of said set of genes,
g) for each gene transcript of said set of genes, converting the level of each gene transcript obtained at step f) into a fractional rank by dividing the rank of the gene having said level of said gene transcript by the number of genes from said set of genes,
h) isolating from the fractional ranks obtained at step g) the fractional rank obtained for the genes of the subset of genes (1) and computing their mean fractional rank (MFR-subset(1) or MFR-positive),
i) isolating from the fractional ranks obtained at step g) the fractional rank obtained for the genes of the subset of genes (2) and computing their mean fractional rank (MFR-subset(2) or MFR-negative), and
e) computing a second deR score as MFR-subset(1) less MFR-subset(2), and
k) comparing said first deR score with said second deR score, wherein an observed deviation between said first and said second deR score is indicative of a progression or a regression of said cancer.

According to another of its objects, the disclosure relates to a method for monitoring a progression or a regression of a cancer in a subject presumed or known to have a TEAD-active cancer, the method comprising the use of a set of gene transcripts from a set of genes (B), and the method comprising at least the steps of:
a) obtaining, from a first biological sample obtained from said subject at a first time, a level of each gene transcript of said set of genes,
b) multiplying each obtained level of each gene transcript of said set of genes obtained at step a) by a coefficient associated with each gene, to obtain, for each gene, a product (Pgeneᵢ wherein gene; refers to a gene listed in said set of genes);
c) summing the products Pgeneᵢ (ΣPgeneᵢ) obtained at step b) and adding a constant for obtaining a first (S) score: (ΣPgeneᵢ+constant),
d) obtaining, from a second biological sample obtained from said subject at a second time, subsequent to the first time, a level of each gene transcript of said set of genes,
e) multiplying each obtained level of each gene transcript of said set of genes by a coefficient associated with each gene, to obtain, for each gene, a product (Pgeneᵢ) wherein gene; refers to a gene listed in said set of genes;
f) summing the products Pgeneᵢ (ΣPgeneᵢ) obtained at step e) and adding a constant for obtaining a second (S) score: (ΣPgeneᵢ+constant), and
g) comparing said first (S) score with said second (S) score, wherein an observed deviation between said first and said second (S) score is indicative of a progression or a regression of said cancer,
wherein the coefficient(s) and the constant used at steps b), c), e), and f) are previously obtained by a stepwise multiple linear regression analysis correlating (i) levels of said gene transcripts previously obtained in a third biological sample obtained from a subject having said cancer with (ii) levels of gene transcripts of a TEAD-500 signature obtained in a fourth biological sample obtained from said subject, the fourth biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed thereafter in Table 2. The subject from whom are taken the third and fourth biological samples may be the same or may be different from the subject from whom are taken the biological samples for computing the (S) scores. The third and fourth biological samples are representative of the cancer for which the method according to the disclosure is considered.

According to another of its objects, the disclosure relates to a method for monitoring a response of a subject presumed to or known to have a TEAD-active cancer to a TEAD-inhibitor treatment, the method comprising the use of a set of gene transcripts from a set of genes (A), and the method comprising at least the steps of:
a) obtaining, from a first biological sample obtained from said subject before administration of a TEAD-inhibitor treatment, a level of each gene transcript of said set of genes,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a first deR score as MFR-subset(1) less MFR-subset(2),
f) obtaining, from a second biological sample obtained from said subject after administration of a TEAD-inhibitor treatment, a level of each gene transcript of said set of genes,
g) for each gene transcript of said set of genes, converting the level of each gene transcript obtained at step f) into a fractional rank by dividing the rank of the gene having said level of said gene transcript by the number of genes from said set of genes,
h) isolating from the fractional ranks obtained at step g) the fractional rank obtained for the genes of the subset of genes (1) and computing their mean fractional rank (MFR-subset(1) or MFR-positive),
i) isolating from the fractional ranks obtained at step g) the fractional rank obtained for the genes of the subset of genes (2) and computing their mean fractional rank (MFR-subset(2) or MFR-negative), and
e) computing a second deR score as MFR-subset(1) less MFR-subset(2), and
k) comparing said first deR score with said second deR score, wherein an observed deviation between said first and said second deR score is indicative of an effective or ineffective TEAD-inhibitor treatment.

According to another of its objects, the disclosure relates to a method for monitoring a response of a subject presumed or known as having a TEAD-active cancer to a TEAD-inhibitor treatment, the method comprising the use of a set of gene transcripts from a set of genes (B), and the method comprising at least the steps of:
a) obtaining, from a first biological sample obtained from said subject before administration of a TEAD-inhibitor treatment, level of each gene transcript of said set of genes,
b) multiplying each obtained level of each gene transcript of said set of genes obtained at step a) by a coefficient associated with each gene, to obtain, for each gene, a product (Pgeneᵢ) wherein gene; refers to a gene listed in said set of genes;
c) summing the products Pgeneᵢ (ΣPgeneᵢ) obtained at step b) and adding a constant for obtaining a first (S) score: (ΣPgeneᵢ+constant),
d) obtaining, from a second biological sample obtained from said subject after administration of a TEAD-inhibitor treatment, a level of each gene transcript of said set of genes,
e) multiplying each obtained level of each gene transcript of said set of genes by a coefficient associated with each gene, to obtain, for each gene, a product (Pgeneᵢ) wherein gene; refers to a gene listed in said set of genes;
f) summing the products Pgeneᵢ (ΣPgeneᵢ) obtained at step e) and adding a constant for obtaining a second (S) score: (ΣPgeneᵢ+constant), and
g) comparing said first (S) score with said second (S) score, wherein an observed deviation between said first and said second (S) score is indicative of an effective or ineffective TEAD-inhibitor treatment,
wherein the coefficient(s) and the constant used steps b), c), e) and f) are previously obtained by stepwise multiple linear regression analysis for correlating (i) levels of said gene transcripts previously obtained in a third biological sample obtained from a subject having said cancer with (ii) levels of gene transcripts of a TEAD-500 signature obtained in a fourth biological sample obtained from said subject, the fourth biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed thereafter in Table 2. The subject from whom are taken the third and fourth biological samples may be the same or may be different from the subject from whom are taken the biological samples for computing the (S) scores. The third and fourth biological samples are representative of the cancer for which the method according to the disclosure is considered.

According to another of its objects, the disclosure relates to a method for screening a TEAD-inhibitor candidate for inhibiting a TEAD activity, the method comprising the use of a set of gene transcripts from a set of genes (A), and the method comprising at least the steps of:
a) obtaining, from a first biological sample obtained from a supernatant of a cell culture, the first biological sample being obtained before contacting the cell culture with said TEAD-inhibitor candidate compound, a level of each gene transcript of said set of genes,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a first deR score as MFR-subset(1) less MFR-subset(2),
f) obtaining, from a second biological sample obtained from a supernatant of said cell culture, the second biological sample being obtained after contacting said cell culture with said TEAD-inhibitor candidate compound, a level of each gene transcript of said set of genes,
g) for each gene transcript of said set of genes, converting the level of each gene transcript obtained at step f) into a fractional rank by dividing the rank of the gene having said level of said gene transcript by the number of genes from said set of genes,
h) isolating from the fractional ranks obtained at step g) the fractional rank obtained for the genes of the subset of genes (1) and computing their mean fractional rank (MFR-subset(1) or MFR-positive),
i) isolating from the fractional ranks obtained at step g) the fractional rank obtained for the genes of the subset of genes (2) and computing their mean fractional rank (MFR-subset(2) or MFR-negative), and
e) computing a second deR score as MFR-subset(1) less MFR-subset(2), and
k) comparing said first deR score with said second deR score, wherein an observed deviation between said first and said second deR score is indicative of an effective or ineffective TEAD-inhibitor candidate compound for inhibiting a TEAD activity.

A suitable cell for a cell culture in accordance with the disclosure may be a TEAD-active cell.

According to another of its objects, the disclosure relates to a method for screening a TEAD-inhibitor candidate for inhibiting a TEAD activity, the method comprising the use of a set of gene transcripts from a set of genes (B), and the method comprising at least the steps of:
a) obtaining, from a first biological sample obtained from a supernatant of a cell culture, the supernatant being obtained before contacting the cell culture with said TEAD-inhibitor candidate compound, a level of each gene transcript of said set of genes,
b) multiplying each obtained level of each gene transcript of said set of genes obtained at step a) by a coefficient associated with each gene, to obtain, for each gene, a product (Pgeneᵢ wherein gene; refers to a gene listed in said set of genes);
c) summing the products Pgeneᵢ (ΣPgeneᵢ) obtained at step b) and adding a constant for obtaining a first (S) score: (ΣPgeneᵢ+constant),
d) obtaining, from a second biological sample obtained from a supernatant of said cell culture, the supernatant being obtained after contacting said cell culture with said TEAD-inhibitor candidate compound, a level of each gene transcript of said set of genes,
e) multiplying each obtained level of each gene transcript of said set of genes by a coefficient associated with each gene, to obtain, for each gene, a product (Pgeneᵢ) wherein gene; refers to a gene listed in said set of genes;
f) summing the products Pgeneᵢ (ΣPgeneᵢ) obtained at step e) and adding a constant for obtaining a second (S) score: (ΣPgeneᵢ+constant), and
g) comparing said first (S) score with said second (S) score, wherein an observed deviation between said first and said second (S) score is indicative of an effective or ineffective TEAD-inhibitor treatment,
wherein the coefficient(s) and the constant used at steps b), c), e) and f) are previously obtained by stepwise multiple linear regression analysis for correlating (i) levels of said gene transcripts previously obtained in a third biological sample obtained from a supernatant of cell culture with (ii) levels of gene transcripts of a TEAD-500 signature obtained in a fourth biological sample obtained from said cell culture, the fourth biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed thereafter in Table 2. The cell culture from which are taken the third and fourth biological samples may be the same or may be different from the cell culture from which are taken the biological samples for computing the (S) scores.

A suitable cell for a cell culture in accordance with the disclosure may be a TEAD-active cell.

In some embodiments, the set of gene transcripts may be obtained from isolated extracellular vesicles.

In some embodiments, the cancer may be selected among mesothelioma, adrenocortical carcinoma, bladder urothelial carcinoma, breast invasive carcinoma, cervical squamous cell carcinoma and endocervical adenocarcinoma, cholangiocarcinoma, consensus molecular subtypes 1 of colorectal cancer, consensus molecular subtypes 2 of colorectal cancer, consensus molecular subtypes 3 of colorectal cancer, consensus molecular subtypes 4 of colorectal cancer, colon adenocarcinoma, lymphoid neoplasm diffuse large b-cell lymphoma, esophageal carcinoma, glioblastoma multiforme, head and neck squamous cell carcinoma, kidney renal clear cell carcinoma, kidney renal papillary cell carcinoma, brain lower grade glioma, liver hepatocellular carcinoma, lung adenocarcinoma, lung squamous cell carcinoma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, and uterine carcinosarcoma.

In some embodiments, the cancer may be a mesothelioma.

In some embodiments, the cancer may be a colorectal cancer.

According to another of its objects, the disclosure relates to a kit comprising a solid support comprising a panel of nucleic acid for obtaining gene transcript levels of a set of gene transcripts as disclosed herein.

### DESCRIPTION OF THE FIGURES

FIG. 1: represents the process from cell treatment to extracellular vesicles (EVs) isolation and analysis.

FIG. 2: represents the TEAD activity in parent cells after a TEAD-inhibitor (TEADi) treatment.

FIG. 3: represents the percentage of the genes from the TEAD-500 signature detected in EVs mRNAs.

FIG. 4: represents the comparison of TEAD signature profiles between extracellular vesicles (EVs) and parental cell in TEADi dose response analysis.

FIG. 5: represents the TEAD-inhibitor (TEADi)-dose effects on TEAD activity in parent cells as measured by a restricted 28-gene TEAD signature in extracellular vesicles (EVs) and in the parent cells.

FIG. 6: represents the TEAD activity measured in extracellular vesicles (EVs) using curated markers (n=4) and in parent cells using the TEAD-500 signature (n=500).

### DETAILED DESCRIPTION

### Definitions

Unless otherwise defined herein, scientific and technical terms used in connection with the present disclosure shall have the meanings that are commonly understood by those of ordinary skill in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill in the art with a general dictionary of many of the terms used in this disclosure. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular.

Units, prefixes, and symbols are denoted in their International System Units (Système International des Unités (SI)) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

All publications and other references mentioned herein are incorporated by reference in their entirety. Although a number of documents are cited herein, this citation does not constitute an admission that any of these documents forms part of the common general knowledge in the art.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that wherever aspects are described herein with the language “comprising,” otherwise analogous aspects described in terms of “consisting of” and/or “consisting essentially of” are also provided.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

By “obtained”, “purified” and “Isolated”, it is meant, when referring to a biological sample, a gene transcript, a population or sub-population of a gene transcripts, a set of gene transcripts, an extracellular vesicle, a population or a sub-population of extracellular vesicles, that the indicated element has been separated from other substances or components that were originally present in a mixture or from its natural environment. The term “purified” as used herein in particular means at least 75%, 85%, 95%, or 98% by weight, of elements of the same type are present.

As used herein, the term “subject” or “patient” denotes a mammal, such as a rodent, a feline, a canine, and a primate. In particular, a subject according to the disclosure is a human. A subject in need thereof may be a subject known or presumed to have a cancer. In some embodiments, a subject in need thereof may be a subject known or presumed to have a TEAD-active cancer. In some embodiments, a subject may be an animal model of a TEAD-active cancer.

“Biomarker” intends to refer a biological molecule or a set of biological molecules, for example a RNA molecule or a set of RNA molecules, that is differentially present, increased or decreased, in a biological sample obtained from a subject or a group of subjects having a first phenotype, e.g., having a disease such as a cancer or a TEAD-active cancer, as compared to a biological sample from a subject or a group of subjects having a second phenotype, e.g., not having the disease or not having a TEAD-active cancer. In use, the biomarker is isolated from the subject.

Signature, gene signature, and transcriptional signature. Those terms and expression as used herein intend to refer to a pattern of levels of transcription of a set of genes, or a pattern of levels of gene transcripts, which can be associated with a TEAD-active cancer or tumor. A transcriptional signature may be obtained by measuring the levels of the gene transcripts of the disclosure in a biological sample. In the biological sample, one may measure (or obtain) the levels of all or part of the gene transcripts susceptible to be present in the biological sample.

Herein, “gene transcript”, “transcription” and “expression of a gene” are used interchangeably to refer to the production of RNA from a gene. The transcription or expression of a gene may increase or decrease resulting in an increase or decrease of the production of RNA.

Expression level of a gene. As used herein, the terms “expression level of a gene” refers to the level of RNA transcripted (or gene transcript) from any given gene. A level of a gene transcript obtained by RNA-seq may be expressed as Fragments Per Kilobase of transcript per Million mapped reads (FPKM).

Level of gene transcript. As used herein, the terms “level of gene transcript” intends to refer to a measure of how much a gene is expressed or transcripted in a biological sample. It reflects the amount of RNA produced from a gene by transcription. A level of gene transcript may be obtained or measured by various methods known in the art, such as reverse transcription polymerase chain reaction (RT-PCR), microarrays, and RNA sequencing (RNA-seq). In the disclosure, the terms “a level of gene transcript” and “a gene transcript level” are used interchangeably. In the disclosure, the terms “levels of gene transcripts” or “gene transcript levels” are used interchangeably.

FPKM (Fragments Per Kilobase of transcript per Million mapped reads) is a measure of gene expression level (or of a gene transcript level) that takes into account the number of RNA sequencing reads mapped to a particular gene and the gene length, normalized by the total number of reads in the sample

Positive effector. A positive effector is a gene of which the gene transcript level correlates positively with TEAD activity. It increases when the TEAD pathway is somehow activated or decreases when the TEAD pathway is inhibited. A positive effector is not necessarily expected to respond in both directions of TEAD modulation. For example, a gene of which the normal expression in a tissue is null, may be seen increased upon TEAD activation but cannot be expected to decrease upon TEAD inhibition. Even at higher normal levels of expression, a positive effector gene may not necessarily be sensitive to both TEAD activation and inhibition.

Negative effector. A negative effector is a gene of which the gene transcript level correlates negatively with TEAD activity. It decreases upon TEAD activation or increases with TEAD inhibition. Like a positive effector, a negative effector is not necessarily expected to respond to TEAD modulation in both directions. For example, a gene of which the normal expression in a tissue is null, may be seen increased upon TEAD-pathway inhibition but cannot be expected to decrease upon TEAD activation. Even at higher normal levels of expression, a negative effector gene may be sensitive to TEAD activation, but immune to TEAD inhibition, or vice versa.

The TEAD-500 signature gene list. A list of 430 to 482 most significant positive or negative gene effectors of TEAD modulation. These genes were selected by differential expression analyses of 18 published expression array datasets. Some of the genes are direct targets of TEAD transcription factors, meaning that they present a TEAD-recognition motif in their promoter, but most of these genes are subject to secondary or indirect regulation by the TEAD pathway. The “TEAD-500” signature gene list consists of 482 genes to about 90% of the 482 genes. Dropouts up to at least 10% only negligibly influence its score. This is one of the reasons why TEAD-500 is highly robust. A TEAD-500 signature is disclosed in Calvet et al. (BMC Cancer, 2022, 22:639, doi.org/10.1186/s12885-022-09686-y).

Extracellular Vesicles (EVs) intends to refer to vesicles shed from cells such as exosomes, microvesicles (or ectosomes), apoptotic bodies, oncosomes, exosomes-like vesicles. The EVs are cell-derived small membrane vesicles secreted by cells that convey biological messages, either by surface-to-surface interaction or by shuttling bioactive molecules to a recipient cell's cytoplasm. The EVs released by cells harbor the cargos of their original cells and could be a good tool to follow drug activity.

“Sample” or “biological sample” intends to refer to a biological material obtained (or purified or isolated) from a subject or from a cell culture. The biological sample may contain any biological material suitable for detecting the biomarker, i.e., a gene transcript, and may comprise cellular and/or non-cellular material, such as extracellular vesicles. A biological sample may be a body fluid sample. A body fluid sample may be isolated from any suitable biological fluid such as, for example, blood, blood plasma (plasma), blood serum (serum), saliva, urine, or cerebral spinal fluid (CSF). In some embodiments, a biological sample is a blood plasma (plasma), a blood serum (serum) sample, a saliva sample, or a urine sample.

A “value of reference” or a “threshold value” intends to refer, according to the context, to a level of a gene transcript, or to levels of gene transcripts, or to a transcriptional signature, or to a deR score, or to a (S) score, which is indicative of a particular disease state, phenotype, such as cancer, or lack thereof, as well as combinations of disease states, phenotypes, or lack thereof, in a concerned subject.

TEAD-active cancer. A “TEAD-active cancer” as used herein intends to refer to a cancer for which the cancer cells have a dysregulated TEAD-pathway resulting in oncogenic transcription, cancer cells/tumor development or evolution, and which can respond to a treatment with a TEAD-inhibitor (TEADi).

TEAD signature. “TEAD signature” or “TEAD-500 signature” as used herein intends to refer to a transcriptional signature obtained by measure of the expression levels of the genes of a set of genes comprising any of 220 to 249 of positive effector genes and any of 210 to 233 of negative effector genes as listed herein in Table 2. By extension, depending on the context, a “TEAD signature” may refer to the associated set of genes.

Scoring. TEAD-500 scoring (herein referred to as “TEAD score”) is a procedure of calculating the activity of the TEAD pathway in a sample, on a continuous scale of values. In some embodiments, the score is based on the mean fractional ranks of the transcript levels of the positive and negative effectors.

Fractional rank. In an array of values such as the gene transcripts levels in a transcriptome of a set of genes, each value is replaced by its rank, with the smallest value taking the rank 1. The fractional rank of a gene equals its rank (determined by the level of its gene transcript) divided by the number of genes in the set (maximum rank).

Difference of effector ranks (deR). The levels of the transcripts of the TEAD-500 signature (from 430 to 482 or 500 gene transcripts) are transformed into fractional ranks,

Calling (binning) the TEAD activity status. This is a binary transformation of the continuous deR score of TEAD activity into two discrete status values: “active” or “inactive”. In some embodiments, we empirically set the deR threshold to 0.055. This value was chosen from an experiment with human mesothelioma cell lines. We observed that cell lines with deR score less than 0.055 did not respond to YAP1-siRNA treatment whereas cell lines with higher scores stopped growing when YAP1 was so knocked-out. The threshold of response may be different for different tissues or treatments. The score value of 0.055 approximately corresponds to the 88^{th} percentile of the scores observed in cancer cells of the Cancer Genome Atlas (TCGA) cohort (all indications pooled). If the deR score of TEAD-500 is greater than 0.055, TEAD is called active, otherwise, inactive.

Mathematical normalization. Mathematical normalization is a process of transforming data or values into a standard or common scale.

TEAD-pathway inhibitor or TEAD-Inhibitor (TEADI). As used herein, a TEAD-pathway inhibitor is any small or large molecule compound that inhibits the HIPPO-YAP/WWTR1/TEAD pathway.

“Administer” or “administering,” as used herein refers to delivering to a subject a composition described herein. The composition can be administered to a subject using methods known in the art. In particular, the composition can be administered intravenously, subcutaneously, intramuscularly, intradermally, or via any mucosal surface, e.g., orally, sublingually, buccally, nasally, rectally, vaginally or via pulmonary route. In some embodiments, the administration is intravenous. In some embodiments, the administration is subcutaneous.

The terms “treat”, “treatment”, or “therapy” refers to the administration of a compound or a composition according to the disclosure with the purpose to cure, heal, alleviate, relieve, after, remedy, ameliorate, improve, or affect a disorder, the symptoms of the condition, or to prevent or delay the onset of the symptoms, complications, or otherwise arrest or inhibit further development of the disorder in a statistically significant manner. More particularly, “treating” or “treatment” includes any approach for obtaining beneficial or desired results in a subject's cancer condition. Beneficial or desired clinical results can include, but are not limited to, alleviation or amelioration of one or more cancer symptoms or conditions, diminishment or reduction of the extent of a cancer disease or of a cancer symptom, stabilizing, i.e., not worsening, the state of a cancer disease or of a cancer symptom, prevention of a cancer disease or of a cancer symptom's spread, delay or slowing of cancer disease or cancer symptom progression, amelioration or palliation of the cancer disease state, diminishment of the reoccurrence of cancer disease, and remission, whether partial or total and whether detectable or undetectable. In other words, “treatment” as used herein includes any cure, amelioration, or reduction of a cancer disease or symptom. A “reduction” of a symptom or a disease means decreasing of the severity or frequency of the disease or symptom, or elimination of the disease or symptom.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” the list of items “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

### DETAILED DESCRIPTION

### Sets of Gene Transcripts

In some embodiments, a set of gene transcripts (A) from a set of genes may consist in a subset of genes (1) consisting of ADM, AXL, BIRC5, CDV3, CRIM1, CTGF, CYR61, FSTL1, GADD45A, KRT8, LMNB2, MATN2, PKP4, RND3, RPS24, SEC14L1, SGK1, SLC25A3, SLC3A2, TNFRSF12A, TPM1, TPX2, and TUBB6 and of a subset of genes (2) consisting of CTSB, FTH1, SQSTM1, TCF25, and UBC.

A set of gene transcripts (A) is obtained from a set of genes consisting of a first subset of genes (1) and of a second subset of genes (2). The first subset of genes (1) is consisting of ADM, AXL, BIRC5, CDV3, CRIM1, CTGF, CYR61, FSTL1, GADD45A, KRT8, LMNB2, MATN2, PKP4, RND3, RPS24, SEC14L1, SGK1, SLC25A3, SLC3A2, TNFRSF12A, TPM1, TPX2, and TUBB6. The second subset of genes (2) is consisting of CTSB, FTH1, SQSTM1, TCF25, and UBC.

In some embodiments, a set of gene transcripts (B) may consist in at least one gene transcript from a set of genes consisting of DLC1, AKAP2, CANX, SAFB2, EIF4H, NDUFS5, SEPT9, and EIF4A1.

In some embodiments, a set of gene transcripts (B) may consist in at least 2, 3, 4, 5, 6, 7, or in consisting of 8 gene transcripts from said set of genes.

In some embodiments, a set of gene transcripts (B) may consist in at least two gene transcripts from the set of genes consisting of DLC1, AKAP2, CANX, SAFB2, EIF4H, NDUFS5, SEPT9, and EIF4A1.

In some embodiments, a set of gene transcripts (B) may consist in a set of genes consisting of at least two gene transcripts, one being a gene transcript from gene DLC1 and the at least second gene transcript being from a set of genes consisting of AKAP2, CANX, SAFB2, EIF4H, NDUFS5, SEPT9, and EIF4A1.

In some embodiments, a set of gene transcripts (B) may consist in a set of gene transcripts from a set of genes consisting of DLC1, and AKAP2, and optionally of at least one gene selected from CANX, SAFB2, EIF4H, NDUFS5, SEPT9, and EIF4A1.

In some embodiments, a set of gene transcripts (B) may consist in a set of gene transcripts from a set of genes consisting of DLC1, AKAP2, and CANX, and optionally of at least one gene selected from SAFB2, EIF4H, NDUFS5, SEPT9, and EIF4A1.

In some embodiments, a set of gene transcripts (B) may consist in a set of gene transcripts from a set of genes consisting of DLC1, AKAP2, CANX, and SAFB2, and optionally of at least one gene selected from EIF4H, NDUFS5, SEPT9, and EIF4A1.

In some embodiments, a set of gene transcripts (B) may consist in a set of gene transcripts from a set of genes consisting of DLC1, AKAP2, CANX, SAFB2, and EIF4H, and optionally of at least one gene selected from NDUFS5, SEPT9, and EIF4A1.

In some embodiments, a set of gene transcripts (B) may consist in a set of gene transcripts from a set of genes consisting of DLC1, AKAP2, CANX, SAFB2, EIF4H, and NDUFS5, and optionally of at least one gene selected from SEPT9 and EIF4A1.

In some embodiments, a set of gene transcripts (B) may consist in a set of gene transcripts from a set of genes consisting of DLC1, AKAP2, CANX, SAFB2, EIF4H, NDUFS5, and SEPT9, and optionally of EIF4A1.

In some embodiments, a set of gene transcripts (B) may consist in a gene transcript from a gene DLC1.

In some embodiments, a set of gene transcripts (B) may consist in a set of gene transcripts from a set of genes consisting of DLC1 and AKAP2.

In some embodiments, a set of gene transcripts (B) may consist in a set of gene transcripts from a set of genes consisting of DLC1, AKAP2, and CANX.

In some embodiments, a set of gene transcripts (B) may consist in a set of gene transcripts from a set of genes consisting of DLC1, AKAP2, CANX, and SAFB2.

In some embodiments, a set of gene transcripts (B) may consist in a set of gene transcripts from a set of genes consisting of DLC1, AKAP2, CANX, SAFB2, and EIF4H.

In some embodiments, a set of gene transcripts (B) may consist in a set of gene transcripts from a set of genes consisting of DLC1, AKAP2, CANX, SAFB2, EIF4H and NDUFS5.

In some embodiments, a set of gene transcripts (B) may consist in a set of gene transcripts from a set of genes consisting of DLC1, AKAP2, CANX, SAFB2, EIF4H, NDUFS5 and SEPT9.

In some embodiments, a set of gene transcripts (B) may consist in a set of gene transcripts from a set of genes consisting of DLC1, AKAP2, CANX, SAFB2, EIF4H, NDUFS5, SEPT9 and EIF4A1.

A gene transcript may be an isolated or purified gene transcript.

In some embodiments, a set of gene transcripts may be an isolated or a purified set of gene transcripts.

In some embodiments, a gene transcript may be an RNA molecule.

### Assay/RNA Sequencing

In some embodiments, a level of gene transcript or levels of gene transcripts levels may be obtained.

In some embodiments, a level of a gene transcript may be a nucleic acid expression level, such as an RNA level, e.g., an mRNA level, or a DNA expression level. Any suitable method for determining a nucleic acid expression level (or a gene transcript level) may be used.

In some embodiments, a level of a gene transcript may be measured by a method selected from RNA sequencing (RNA-seq), Next-generation sequencing (NGS), digital PCR, droplet digital PCR (ddPCR), RT-qPCR, qPCR, multiplex qPCR microarray analysis, serial analysis of gene expression (SAGE), whole genome sequencing (WGS), and Mass-ARRAY techniques.

For example, a gene transcript level may be determined using an RNA ACCESS protocol or TRUSEQ RIBO-ZERO00 protocol (ILLUMINA)), RT-qPCR, qPCR, multiplex qPCR or RT-qPCR, microarray analysis, SAGE, Mass-ARRAY techniques, or a combination thereof.

In addition, such methods can include one or more steps that allow one to determine the levels of target RNA in a biological sample, for example, by simultaneously examining the levels of a comparative control RNA sequence of a “housekeeping” gene such as an actin family member. In some embodiments, the sequence of the amplified target cDNA can be determined.

In some embodiments, the gene transcript is an RNA molecule and the level is determined using RNA-seq.

e embodiments, the gene transcript is an mRNA molecule.

RNA-seq allows measuring qualitatively and quantitively genes expression at the transcriptome level and involves sequencing RNA molecules from a sample and mapping them to a reference genome or transcriptome. Different methods for RNA-seq are known in the art (Zhang et al. (2020). Systematic comparison and assessment of RNA-seq procedures for gene expression quantitative analysis. Scientific Reports 10: 20765. www.nature.com/articles/s41598-020-76881-x).

RNA-seq usually involves the preparation of an RNA sequencing library. Standard protocols can be used for preparing an RNA sequencing library. For example, the RNAs may be fragmented and reverse-transcribed into cDNA. Adapters may be added to the cDNA, and the library may be amplified using PCR. Adapters are a short DNA sequences allowing the fragments to be amplified by PCR and providing a barcode or an identifier for the sequencing platform. The RNA library may be then sequenced using any know methods in the art, such as next-generation sequencing to obtain the RNA-seq data.

Usually, RNA-seq data analysis involves the following steps: trimming to eliminate adapter sequences and poor-quality nucleotides, alignment to a reference genome or transcriptome, counting or quantification by assigning the reads to a gene or a transcript, normalization of the sequenced reads, and, very often, differential expression (DE) analysis across conditions.

The levels of gene transcripts may be quantified using software such as Cufflinks or RSEM. The differential expression analysis may be performed using software such as DESeq2 or edgeR.

Optional methods include protocols which examine or detect RNAs, such as target RNAs, in a sample by microarray technologies.

Using nucleic acid microarrays, test and control RNA samples from test and control samples are reverse transcribed and labeled to generate cDNA probes. The probes are then hybridized to an array of nucleic acids immobilized on a solid support. The array is configured such that the sequence and position of each member of the array is known. For example, any of the gene transcripts described herein may be arrayed on a solid support. Hybridization of a labeled probe with a particular array member indicates that the sample from which the probe was derived expresses that gene.

The level of a gene transcript can be measured and quantified using various methods as previously detailed, such as microarray analysis or RNA sequencing (RNA-seq). The level of a gene transcript may be expressed is by using FPKM (Fragments Per Kilobase of transcript per Million mapped reads), a normalized measurement that takes into account the length of the transcript and the total number of reads generated by RNA sequencing.

FPKM is calculated by dividing the number of fragments (or reads) that map to a particular gene transcript by the length of that transcript in kilobases, and then normalizing this value by the total number of mapped fragments in the sample (in millions). This allows for the comparison of expression levels between different genes and different samples.

Other suitable measures of a level of a gene transcript include TPM (Transcripts Per Million) and RPKM (Reads Per Kilobase of transcript per Million mapped reads).

TPM (Transcripts Per Million) is a method for normalizing the level of gene transcripts in RNA sequencing data. TPM takes into account both the length of the gene transcript and the total number of reads generated by RNA sequencing but normalizes the level of a gene transcript by dividing the number of reads that map to a particular gene transcript by the total number of reads in the sample, and then multiplying this value by one million. This allows for the comparison of levels between different gene transcripts and different samples and takes into account differences in gene transcript length and sequencing depth.

RPKM (Reads Per Kilobase of transcript per Million mapped reads) is a method for quantifying the level of a gene transcript in RNA sequencing data. Like FPKM and TPM, RPKM takes into account both the length of the gene transcript and the total number of reads generated by RNA sequencing.

RPKM is calculated by dividing the number of reads that map to a particular gene transcript by the length of that gene transcript in kilobases, and then dividing this value by the total number of mapped reads in the sample (in millions). The resulting value represents the level of the gene transcript in units of reads per kilobase per million mapped reads. This allows for the comparison of levels between different gene transcripts and different samples, while taking into account differences in transcript length and sequencing depth.

In some embodiments, the level of a gene transcript of the disclosure is measured by RNA sequencing (RNA-seq) and is expressed/quantified as FPKM (Fragments Per Kilobase of transcript per Million mapped reads).

### Biological Samples

In some embodiments, the gene transcripts may be obtained from a biological sample or a fraction thereof. A fraction of a biological sample suitable for the disclosure may be an extract of such sample.

A biological sample may be obtained from a subject in need thereof, a cell culture, or an animal model, e.g., an animal model of cancer.

A cell suitable for the disclosure may be a TEAD-active cell. As example of TEAD-active cells suitable for cell culture according to the disclosure, one may cite mesothelioma cells lines, NCI-H226, Mero-14, and SPC212. As example of TEAD-inactive cells suitable as control according to the disclosure one may cite the non-responding colon cell line, HCT116.

Examples of animal model of TEAD-active cancer may be mouse models of colorectal cancer, lung cancer, liver cancer and breast cancer with genetic deletion or mutation of TEADs or their co-factors, or with overexpression or knockdown of TEADs or their co-factors.

A biological sample may be a body fluid sample or a cell culture supernatant.

A body fluid sample may be isolated from a subject in need thereof.

A body fluid sample may be selected from a blood sample, a plasma sample, a urine sample, a cerebrospinal fluid (CSF) sample, a bronchoalveolar fluid sample, a nasal secretion sample, a breast milk sample, a semen sample, and a saliva sample.

In some embodiments, a body fluid sample may be a blood sample.

In some embodiments, a body fluid sample may be a plasma sample.

In some embodiments, a body fluid sample may be a urine sample.

In some embodiments, a body fluid sample may be a saliva sample.

In some embodiments, a biological sample or a fraction thereof may comprise extracellular vesicles.

A body fluid sample may be obtained by any known methods in the art. For example, one may cite needle puncture methods for obtaining blood samples, passive draining, or aspiration for obtaining saliva samples, lumbar puncture for obtaining CSF sample, midstream clean catch method or catheterization for obtaining urine samples, bronchoalveolar lavage methods for obtaining bronchoalveolar samples.

A biological sample is taken in an amount sufficient to contain the necessary biological materials to obtain the gene transcripts of the disclosure. The skilled person knows how to adapt the amount of biological sample according to the nature and the source of the biological sample.

For example, depending on the fluid samples, the volume of the sample may range from about 1 ml, for a saliva sample, to about 250 ml, for a bronchoalveolar sample.

A biological sample, or fraction thereof, suitable for the disclosure may contain extracellular vesicles (EVs).

### Extracellular Vesicles

In some embodiments, the gene transcripts of the disclosure may be obtained from isolated extracellular vesicles. The EVs may be obtained from a biological sample as disclosed herein.

In some embodiments, the gene transcripts may be extracted from extracellular vesicles.

Extracellular vesicles (EVs) are small, membrane-bound vesicles that are released from cells into the extracellular environment. They can be classified into different subtypes, including exosomes, microvesicles (or ectosomes), apoptotic bodies, oncosomes, or exosomes-like, based on their biogenesis, size, and content. EVs have been found to play important roles in intercellular communication, as they can transport various biomolecules such as proteins, lipids, and nucleic acids between cells.

In some embodiments, the disclosure relates to isolated extracellular vesicles comprising a set of gene transcripts as disclosed herein.

In some embodiments, the disclosure relates to isolated extracellular vesicles comprising a set of gene transcripts (A) obtained from a set of genes, the set of genes consists of a first subset of genes (1) and of a second subset of genes (2), the first subset of genes (1) consisting of ADM, AXL, BIRC5, CDV3, CRIM1, CTGF, CYR61, FSTL1, GADD45A, KRT8, LMNB2, MATN2, PKP4, RND3, RPS24, SEC14L1, SGK1, SLC25A3, SLC3A2, TNFRSF12A, TPM1, TPX2, and TUBB6 and the second subset of genes (2) consisting of CTSB, FTH1, SQSTM1, TCF25, and UBC.

In some embodiments, the disclosure relates to isolated extracellular vesicles comprising a set of gene transcripts consisting of at least one gene transcript from a set of genes consisting of DLC1, AKAP2, CANX, SAFB2, EIF4H, NDUFS5, SEPT9, and EIF4A1.

In some embodiments, each extracellular vesicle of a set of extracellular vesicles may comprise all the gene transcripts of a set of gene transcripts described herein.

In some embodiments, each extracellular vesicle of a set of extracellular vesicles may comprise at least a part of the gene transcripts of a set of gene transcripts described herein, and all the extracellular vesicles taken together of the set of EVs comprise all the gene transcripts of the set of gene transcripts.

In some embodiments, the extracellular vesicles may be selected from exosomes, microvesicles (or ectosomes), apoptotic bodies, oncosomes, exosomes-like, and combinations thereof.

In some embodiments, the extracellular vesicles may be isolated from a biological sample, for example a body fluid sample.

In some embodiments, the extracellular vesicles may be purified from a biological sample, for example a body fluid sample.

The biological sample may be as previously detailed.

Various methods are known in the art for isolation and purification of extracellular vesicles (EVs) from a suitable biological sample. As examples of methods, one may cite:

Ultracentrifugation: such method involves centrifugation of a sample at high speeds to separate EVs from other cellular components.

Size exclusion chromatography (SEC): such method involves separation of EVs based on size, using a column packed with beads that allow for the separation of particles based on their hydrodynamic diameter. Another type of SEC may be an ultracentrifugation method using a sucrose gradient.

Immunoaffinity-based methods: such methods involve using antibodies or other ligands that specifically bind to EVs surface markers, such as CD63 or CD9, to isolate and purify EVs from a sample. As example of immunoaffinity-based methods, one may cite membrane-based affinity column methods.

Membrane-based affinity column: such method involves chromatography column using a membrane with an affinity for EVs to selectively capture and purify them from a sample. The membrane contains a ligand that binds to specific EVs surface markers, such as tetraspanins CD63 or CD9, allowing for the isolation of EVs, for example exosomes and microvesicles, with high purity and specificity.

The workflow for using a membrane-based affinity column typically involves binding EVs from a sample to the membrane, washing away unwanted materials, and then eluting the purified EVs from the membrane using a buffer solution.

Microfluidic-based methods: such methods involve the use of microfluidic devices to isolate and purify EVs based on their size, shape, and other physical properties.

Various methods known in the art may be used to isolate EVs and extract the gene transcripts contents. As example, one may cite differential ultracentrifugation methods, methods using size-based filters (e.g. EXOMIR, BIOOSCIENTIFIC), antibody-based capture methods (e.g. IMMUNOBEADS, HANSABIOMED), or polymer-based precipitation reagents methods (e.g. LIFE TECHNOLOGIES, SYSTEM BIOSCIENCES INC.), or spin column-based methods (membrane-base affinity column).

In some embodiments, the EVs may be isolated from a biological sample, for example a blood or urine sample or a supernatant of cultured cells, using a membrane-based affinity column (EXOEASY, MAXI KIT, QIAGEN). A membrane-based affinity column method may comprise the following steps:
Preparing a membrane-based affinity column by immobilizing a specific ligand on the column matrix. A suitable ligand may be an antibody or aptamer that specifically binds to EVs,
Preparing a biological sample by removing cells and cellular debris through centrifugation or filtration for obtaining a supernatant or a filtrate containing the EVs of interest,
Loading the EV-containing sample is onto the prepared column in conditions suitable for the EVs to bind to the immobilized ligand,
Washing the column with a suitable buffer to remove any unbound materials, and
Eluting the EVs with a suitable change of buffer conditions, such as a change of pH or a salt concentration.

In some embodiments, the extracellular vesicles may be passed in a membrane-base affinity column by centrifugation.

The isolated EVs may be quantified and characterized using various techniques known in the art, such as electron microscopy, nanoparticle tracking analysis, or Western blotting.

From the isolated EVs, the gene transcripts may be extracted, isolated and sequenced by any known methods in the art.

A suitable method for extracting and isolating the gene transcripts of the disclosure may at least comprise the steps involved of:
Lysing the EVs and obtaining the released gene transcripts in a lysate,
Loading the lysate in an affinity column, such as a spin column,
Washing the affinity column with a suitable buffer to remove any unbound materials, and
Eluting the gene transcripts.

The quality and quantity of the gene transcripts can be assessed by any known methods in the art, for example using a bioanalyzer or a spectrophotometer.

### Uses and Methods

### Uses

In some embodiments, a set of gene transcripts as disclosed herein may be for use as a biomarker of a TEAD activity.

In some embodiments, a set of gene transcripts as disclosed herein may be for use for measuring or characterizing a TEAD activity of a cancer.

In some embodiments, a set of gene transcripts as disclosed herein may be for use for measuring or characterizing a TEAD activity of a biological sample.

In some embodiments, a set of gene transcripts as disclosed herein may be for use in a TEAD-inhibitor candidate compound screening method.

In some embodiments, a set of gene transcripts as disclosed herein may be for use in a cancer diagnostic method.

A cancer diagnostic method may be selected among a method for characterizing a TEAD activity status of a cancer, a method for measuring a TEAD activity in a biological sample, a method for predicting a response of a cancer to a TEAD-inhibitor treatment, a method for monitoring a response of a cancer to a TEAD-inhibitor treatment, a method for predicting a TEAD-active cancer progression or regression, and a method for monitoring a TEAD-active cancer progression or regression.

In some embodiments, the disclosure relates to a use of a set of gene transcripts as disclosed herein for characterizing a TEAD activity status of a biological sample.

In some embodiments, the disclosure relates to a use of a set of gene transcripts as disclosed herein for characterizing a TEAD activity status of a cancer in a subject in need thereof.

In some embodiments, the disclosure relates to a use of a set of gene transcripts disclosed herein for measuring a TEAD activity in a biological sample.

In some embodiments, the disclosure relates to a use of a set of gene transcripts disclosed herein for measuring a TEAD activity in a body fluid sample from a subject in need thereof.

In some embodiments, the disclosure relates to a use of a set of gene transcripts disclosed herein for measuring a TEAD activity in a cancer cell sample of a cancer in a subject in need thereof.

In some embodiments, the disclosure relates to a use of the set of gene transcripts disclosed herein for predicting a response of a cancer characterized as TEAD-active to a TEAD-inhibitor treatment, in a subject in need thereof.

In some embodiments, the disclosure relates to a use of a set of gene transcripts disclosed herein for monitoring a response of a cancer characterized as TEAD-active to a TEAD-inhibitor treatment, in a subject in need thereof.

In some embodiments, the disclosure relates to a use of a set of gene transcripts disclosed herein for predicting a cancer progression or regression in a subject in need thereof.

In some embodiments, the disclosure relates to a use of a set of gene transcripts disclosed herein for monitoring a cancer progression or regression in a subject in need thereof.

A subject in need thereof may be a subject known or presumed to have a TEAD-active cancer.

A cancer considered in the present disclosure may be a TEAD-active cancer.

In some embodiments, the disclosure relates to a use of a set of gene transcripts disclosed herein for screening a TEAD-inhibitor candidate compound.

In some embodiments, the set of gene transcripts may be obtained from a biological sample, for example as above detailed.

In some embodiments, the set of gene transcripts may be obtained from extracellular vesicles, for example as above detailed.

In some embodiments, the extracellular vesicles comprising a set of gene transcripts of the disclosure may be used for the uses indicated above for the gene transcripts in which the gene transcripts are extracted from the extracellular vesicles.

In some embodiments, a level of each gene transcript of said set of genes is obtained, for example as above detailed.

The obtained gene transcripts levels may characterize a TEAD-active status of a cancer or of a biological sample.

The obtained gene transcripts levels may be a measure of a TEAD-activity of a cancer or of a biological sample.

In some embodiments, the obtained gene transcripts levels may be compared to values of reference.

In some embodiments, an observed deviation with respect to value(s) of reference may be indicative of a cancer being TEAD-active or TEAD-inactive, or of a cancer being responsive or being not responsive to the TEAD-inhibitor treatment, or of an effective or ineffective TEAD-inhibitor treatment, or of a progressing or regressing cancer, or of a TEAD-inhibitor candidate compound being effective or ineffective.

In some embodiments, a transcriptional signature may be obtained from the gene transcripts levels.

The obtained transcriptional signature may be compared to a transcriptional signature of reference.

In some embodiments, an observed deviation between the obtained transcriptional signature and the transcriptional signature of reference is indicative of a cancer being TEAD-active or TEAD-inactive, or of a cancer being responsive or being not responsive to the TEAD-inhibitor treatment, or of an effective or ineffective TEAD-inhibitor treatment, or of a progressing or regressing cancer, or of a TEAD-inhibitor candidate compound being effective or ineffective.

In some embodiments, the obtained gene transcript level may be subject to a mathematical normalization.

When a set of gene transcripts (A) is used, then a mathematical normalization may be used to compute a deR score. A deR score may be computing according to a method comprising the steps of:
a) for each gene transcript of said set of genes, converting the level of each gene transcript into a fractional rank by dividing the rank of the gene having said level of said gene transcript by the number of genes from said set of genes,
b) isolating from the fractional ranks obtained at step a) the fractional rank obtained for the genes of the subset of genes (1) and computing their mean fractional rank (MFR-subset(1) or MFR-positive),
c) isolating from the fractional ranks obtained at step a) the fractional rank obtained for the genes of the subset of genes (2) and computing their mean fractional rank (MFR-subset(2) or MFR-negative), and
d) computing the deR score as MFR-subset(1) less MFR-subset(2).

The ranking of a gene within a set of genes is obtained by attributing to the gene having the lowest level of gene transcript the rank 1, and to the gene having the next highest level of gene transcript the rank 2, and so on until the gene with the highest level of gene transcript is given the highest rank. Genes having gene transcripts with equal levels are given the average rank, e.g., two genes with identical levels of gene transcripts with 0 expression are given the rank 1.5.

When a set of gene transcripts (B) is used then a mathematical normalization computes a (S) score. A (S) score may be computed according to a method comprising the steps of:
a) multiplying each level of each gene transcript of said set of genes by a coefficient associated with each gene, to obtain, for each gene, a product (Pgeneᵢ) wherein gene, refers to a gene listed in said set of genes;
b) summing the products Pgeneᵢ (ΣPgeneᵢ) obtained at step a) and adding a constant for obtaining a (S) score: (ΣPgeneᵢ+constant),
wherein the coefficient(s) and the constant used at steps a) and b) are previously obtained by a stepwise multiple linear regression analysis correlating (i) levels of said gene transcripts previously obtained in a first biological sample with (ii) levels of gene transcripts of a TEAD-500 signature obtained in a second biological sample, wherein the TEAD-500 signature comprises the gene transcripts of a set of genes comprising any of 220 to 249 of genes of a subset of genes (1) and any of 210 to 233 of genes of a subset of genes (2) as disclosed in Table 2 provided thereafter.

In some embodiments, in the methods and uses of the disclosure, the correlation operated with the stepwise multiple linear regression analysis may be carried out between (i) the levels of said gene transcripts and (ii) a TEAD-score obtained with the levels of gene transcripts of a TEAD-500 signature.

Coefficients and constant are specific for a given set of gene transcripts (B), for a given cancer and for a given method used for determining the level of the gene transcripts, and therefore for the given mode used for determining/quantifying the level. Therefore, for a given cancer, coefficients and constant must be computed for each embodiment of a set of gene transcripts (B), e.g., comprising any combination of one to eight of the gene transcripts of a set of gene transcripts (B). For a set of gene transcripts (B), coefficients and constant must be computed for each cancer type.

The calculated coefficients and constant are dependent on the number of gene transcripts used for the set of gene transcripts (B) and on the method used for obtaining the level of the gene transcripts, and therefore of the mode used for determining/quantifying the levels of the transcripts. The method used for obtaining the levels of the gene transcripts of a set of genes (B) is not necessary the same than the one used of measuring the levels of the gene transcripts of a TEAD-500 signature used for the correlation.

Examples of coefficients and constant usable for mesothelioma and for a set of gene transcripts (B) comprising the 8 gene transcripts and for which the levels are determined in FKPM are presented in the Table 1 below:

TABLE 1

| Set of gene transcripts | |
|---|---|
| (B) with 8 genes | Coefficient |
| DLC1 | 0.031 |
| AKAP2 | 0.036 |
| CANX | −0.023 |
| SAFB2 | 0.040 |
| EIF4H | 0.008 |
| NDUFS5 | −0.011 |
| SEPT9 | −0.009 |
| EIF4A1 | 0.005 |
| Constant | −0.155 |

The first and second biological samples may be obtained from a subject or from cultured cells or from an animal model of TEAD-cancer.

The first and second biological samples are representative of a same TEAD-active cancer or of a same cell culture or of a same animal model of TEAD-active cancer. The first and second biological samples are representative of the cancer or of a cell culture or animal model of a TEAD-active cancer for which a use according to the disclosure is considered. A suitable cell for a cell culture in accordance with the disclosure may be TEAD-active cell.

The first sample may be a body fluid sample or a fraction thereof. The second sample may be a cancer cell sample.

The first and second biological samples may be taken from a same subject.

In some embodiments, the subject from whom are taken the first and second biological samples, which are used for computing the coefficients and the constant by a stepwise multiple linear regression analysis may be the same or may be different from the subject for whom is computed a (S) score.

When different, the subject from whom are computed the coefficients and the constant may be a subject of reference, or a group of subjects of reference, known to have a TEAD-active cancer. Since the coefficients and constant are specific for a given cancer, the subject for whom is computed a (S) score must have or must be presumed to have a same type of cancer than the cancer of the subject of reference or group of subjects of reference (i.e., the subject(s) from whom are previously taken the biological and cancer cell samples).

Two types of biological samples are taken from subject(s) of reference: (a) first biological sample(s) known or presumed to comprise EVs, e.g., a body fluid sample or a fraction thereof, and (a) second biological sample(s) from cancer cells of the cancer.

The levels of gene transcripts of a TEAD-500 signature, as detailed thereafter, are obtained from a biological sample of cancer cells of the cancer and the levels of gene transcripts of a set of genes (B) are obtained from a biological sample known to comprise EVs.

A TEAD-500 signature may comprise the gene transcripts of a set of genes comprising any of 220 to 249 of genes of a subset of genes (1) and any of 210 to 233 of genes of a subset of genes (2) as disclosed in Table 2 thereafter.

A stepwise multiple linear regression analysis correlating (i) levels of the gene transcripts of the set of gene transcripts (B) with (ii) levels of gene transcripts of a TEAD-500 signature is carried out to obtain the coefficients and the constant for said cancer and said set of gene transcripts (B). In some embodiments, the correlation operated with the stepwise multiple linear regression analysis may be carried out between (i) the levels of said gene transcripts and (ii) a TEAD-score obtained with the levels of gene transcripts of a TEAD-500 signature.

In some embodiments, the subject from whom are taken the first and second biological samples used for computing the coefficients and the constant by a stepwise multiple linear regression analysis is the same subject for whom is a computed a (S) score. In such situation, the coefficients and constant may be computed as indicated above. (S) scores computed with the coefficients and constant may be computed on different biological samples taken from said subject, for example, at different subsequent times. Such (S) scores may be used to follow the progression or regression of a TEAD-active cancer or the response of a TEAD-active cancer to a TEAD-inhibitor treatment.

In some embodiments, a first biological sample in which the levels of the gene transcripts are obtained may be a body fluid sample, or a fraction thereof, as previously detailed. A second biological sample may be a cancer cell sample.

In some embodiments, the first and second biological samples may be obtained from cultured cells.

A first biological sample from cultured cells may be a cell supernatant.

A second biological sample from cultured cells may be the cultured cells.

In some embodiments, the cell culture from which are taken the first and second biological samples, i.e., the biological samples previously taken, which are used for computing the coefficients and the constant by a stepwise multiple linear regression analysis, may be the same or may be different from the cell culture for which is computed a (S) score.

A suitable cell for a cell culture in accordance with the disclosure may be TEAD-active cell.

In some embodiments, the first and second biological samples may be obtained from an animal model of a TEAD-active cancer.

A first biological sample may be a fluid body sample, or a fraction thereof, as above indicated.

A second biological sample may be a cancer cell sample.

In some embodiments, the animal model of TEAD-active cancer from whom are taken the first and second biological samples, i.e., the biological samples previously taken, which are used for computing the coefficients and the constant by a stepwise multiple linear regression analysis, may be the same or may be different from the animal for whom is computed a (S) score.

In some embodiments, a deR or (S) score be computed as such.

A computed deR or (S) score may be used for characterizing a TEAD activity status of a cancer in a subject in need thereof, or of a biological sample, or for measuring a TEAD activity in a biological sample, for example of a subject in need thereof.

In some embodiments, a computed deR or (S) score be compared to a value of reference, e.g., a deR or (S) score of reference.

A deR or (S) score of reference may be obtained from a subject or a group of subjects of reference or from a cell culture of reference or from animal model of TEAD-active cancer of reference.

Comparison of a computed deR or (S) score to a deR or (S) score of reference may be used to predict the progression or regression of a TEAD-active cancer in a subject, or to predict the response of a TEAD-active cancer of a subject to a TEAD-inhibitor treatment, or may be used for characterizing a TEAD activity status of a cancer in a subject in need thereof or of a biological sample, or for measuring a TEAD activity in a biological sample, or for screening a TEAD-inhibitor candidate compound.

In some embodiments, a computed deR or (S) score lower or higher than a deR or (S) score of reference may be indicative of a cancer being TEAD-active or TEAD-inactive, or of a cancer being responsive or being not responsive to a TEAD-inhibitor treatment, or of an effective or ineffective TEAD-inhibitor treatment, or of a progressing or regressing TEAD-active cancer, or of a TEAD-inhibitor candidate compound being effective or ineffective.

In some embodiments, a computed deR or (S) score lower than a deR or (S) score of reference may be indicative of a cancer being TEAD-inactive, or of a cancer being responsive to a TEAD-inhibitor treatment, or of an effective TEAD-inhibitor treatment, or of a regressing TEAD-active cancer, or of a TEAD-inhibitor candidate compound being effective.

In some embodiments, a computed deR or (S) score higher than a deR or (S) score of reference may be indicative of a cancer being TEAD-active, or of a cancer being not responsive to a TEAD-inhibitor treatment, or of an ineffective TEAD-inhibitor treatment, or of a progressing TEAD-active cancer, or of a TEAD-inhibitor candidate compound being ineffective.

In some embodiments, when the (S) score may be greater than the value of reference of about 0.055, then the cancer may be characterized as being TEAD-active, and when the (S) score may be less than or equal to about the value of reference of 0.055, then the cancer may be characterized as being TEAD-inactive.

In some embodiments, when the (S) score may be greater than the value of reference of about 0.055, then the cancer may be predicted as being responsive to a TEAD-inhibitor treatment, and when the (S) score may be less than or equal to the value of reference of about 0.055, then the cancer may be characterized as being non-responsive to a TEAD-inhibitor treatment.

In some embodiments, when the (S) score may be greater than the value of reference of about 0.055, then the TEAD-inhibitor treatment may be observed as being ineffective against the TEAD-active cancer, and when the (S) score may be less than or equal to the value of reference of about 0.055, then the TEAD-inhibitor treatment may be observed as being effective against the TEAD-active cancer.

In some embodiments, when the (S) score may be greater than the value of reference of about 0.055, then the TEAD-active cancer may be predicted or detected as being progressing, and when the (S) score may be less than or equal to the value of reference of about 0.055, then the TEAD-active cancer may be predicted or detected as being regressing.

In some embodiments, when the (S) score may be greater than the value of reference of about 0.055, then the TEAD-inhibitor candidate compound may be ineffective for inhibiting a TEAD-activity, and when the (S) score may be less than or equal to the value of reference of about 0.055, then the TEAD-inhibitor candidate compound may be effective for inhibiting a TEAD-activity.

In some embodiments, the reference value may be a first deR or (S) score and the deR or (S) score to be compared to said value of reference may be a second deR or (S) score subsequently measured to the first deR or (S) score.

The first and second deR or (S) scores may be computed from biological samples taken from a same subject or from a same cells culture.

Comparison of first deR or (S) score to second deR or (S) score, or even to further subsequent deR or (S) scores, may be used to monitor the progression or regression of a TEAD-active cancer in a subject or to monitor the response of a TEAD-active cancer of a subject to a TEAD-inhibitor treatment, or to screen a TEAD-inhibitor candidate compound.

In some embodiments, a second deR or (S) score lower than a first deR or (S) score may be indicative of a TEAD-activity inhibition.

In some embodiments, a first deR or (S) score may be measured in a biological sample obtained from a subject known to or presumed to have a TEAD-active cancer and a second deR or (S) score may be measured in a second biological sample obtained from said subject subsequently to said first biological sample, and wherein a second deR or (S) score lower than a first deR or (S) score may be indicative of a regressing cancer in said subject.

In some embodiments, a first deR or (S) score may be measured in a biological sample obtained from a subject known to or presumed to have a TEAD-active cancer and a second deR or (S) score may be measured in a second biological sample obtained from said subject subsequently to said first biological sample, and wherein a second deR or (S) score higher than a first deR or (S) score may be indicative of a progressing cancer in said subject.

In some embodiments, a first deR or (S) score may be measured in a first biological sample obtained from a subject known to or presumed to have a TEAD-active cancer before administration of a TEAD-inhibitor treatment and a second deR or (S) score may be measured in a second biological obtained from said subject after administration of said TEAD-inhibitor treatment, and wherein a second deR or (S) score lower than a first deR or (S) score may be indicative of an effective TEAD-inhibitor treatment in said subject.

In some embodiments, a first deR or (S) score may be measured in a first biological sample obtained from a subject known to or presumed to have a TEAD-active cancer before administration of a TEAD-inhibitor treatment and a second deR or (S) score may be measured in a second biological obtained from said subject after administration of said TEAD-inhibitor treatment, and wherein a second deR or (S) score higher than a first deR or (S) score may be indicative of an ineffective TEAD-inhibitor treatment in said subject.

In some embodiments, a first deR or (S) score may be measured in a first biological sample obtained from an animal model of a TEAD-active cancer or from a supernatant of a cell culture, the first biological sample being obtained before contacting the cell culture with a TEAD-inhibitor candidate compound or before administering the animal model of a TEAD-active cancer with a TEAD-inhibitor candidate compound and a second deR or (S) score may be measured in a second biological sample obtained from said animal model of a TEAD-active cancer or from a supernatant of said cell culture, the second biological sample being obtained after contacting said cell culture with a TEAD-inhibitor candidate compound or after administering the animal model of a TEAD-active cancer with a TEAD-inhibitor candidate compound, and wherein a second deR or (S) score lower than a first deR or (S) score may be indicative of an effective TEAD-inhibitor candidate compound.

In some embodiments, a first deR or (S) score may be measured in a first biological sample obtained an animal model of a TEAD-active cancer or from from a supernatant of a cell culture, the first biological sample being obtained before contacting the cell culture with a TEAD-inhibitor candidate compound or before administering the animal model of a TEAD-active cancer with a TEAD-inhibitor candidate compound and a second deR or (S) score may be measured in a second biological sample obtained from said animal model of a TEAD-active cancer or from a supernatant of said cell culture, the second biological sample being obtained after contacting said cell culture with a TEAD-inhibitor candidate compound or after administering the animal model of a TEAD-active cancer with a TEAD-inhibitor candidate compound, and wherein a second deR or (S) score higher than a first deR or (S) score may be indicative of an ineffective TEAD-inhibitor candidate compound.

A suitable cell for a cell culture in accordance with the disclosure may be a TEAD-active cell.

### Method

In some embodiments the disclosure relates to a method for characterizing or measuring a TEAD activity status of a cancer in a subject in need thereof, the method comprising the use of a set of gene transcripts from a set of genes (A), and the method comprising at least the steps of:
a) obtaining, from a biological sample obtained from said subject, a level of each gene transcript of said set of genes,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a deR score as MFR-subset(1) less MFR-subset(2).

In some embodiments the disclosure relates to a method for characterizing or measuring a TEAD activity status of a cancer in a subject in need thereof, the method comprising the use of a set of gene transcripts from a set of genes (B), and the method comprising at least the steps of:
a) obtaining, from a biological sample obtained from said subject, a level of each gene transcript of said set of genes,
b) multiplying each obtained level of each gene transcript of said set of genes by a coefficient associated with each gene, to obtain, for each gene, a product (Pgeneᵢ) wherein gene; refers to a gene listed in said set of genes;
c) summing the products Pgeneᵢ (ΣPgeneᵢ) obtained at step b) and adding a constant for obtaining a (S) score: (ΣPgeneᵢ+constant),
wherein the coefficient(s) and the constant used at steps b) and c) may be previously obtained by a stepwise multiple linear regression analysis correlating (i) levels of said gene transcripts previously obtained in a first biological sample obtained from a subject having said cancer with (ii) levels of gene transcripts of a TEAD-500 signature obtained in a second biological sample from said subject, the second biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed in Table 2.

In some embodiments, the correlation operated with the stepwise multiple linear regression analysis may be carried between (i) the levels of said gene transcripts and (ii) a TEAD-score obtained with the levels of gene transcripts of a TEAD-500 signature.

As above indicated, the subject from whom are taken the first and second biological samples may be the same or may be different from the subject from whom is taken a biological sample for computing a (S) score. The first and second biological samples are representative of the cancer for which a method according to the disclosure is considered.

In some embodiments, the deR or (S) score may be greater than a value of reference, for example of about 0.055 for a (S) score, then the cancer may be characterized as being TEAD-active, and when the deR or (S) score may be less than or equal to a value of reference, for example of about 0.055 for a (S) score, then the cancer may be characterized as being TEAD-inactive.

In some embodiments the disclosure relates to a method for monitoring a progression or a regression a cancer in a subject presumed or known to have a TEAD-active cancer, the method comprising the use of a set of gene transcripts from a set of genes (A), and the method comprising at least the steps of:
a) obtaining, from a first biological sample obtained from said subject at a first time, a level of each gene transcript of said set of genes,
b) for each gene transcript of said set of genes, converting the level of each gene transcript obtained at step a) into a fractional rank by dividing the rank of said gene by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a first deR score as MFR-subset(1) less MFR-subset(2),
f) obtaining, from a second biological sample obtained from said subject at a second time, subsequent to the first time, a level of each gene transcript of said set of genes,
g) for each gene transcript of said set of genes, converting the level of each gene transcript obtained at step f) into a fractional rank by dividing the rank of the gene having said level of said gene transcript by the number of genes from said set of genes,
h) isolating from the fractional ranks obtained at step g) the fractional rank obtained for the genes of the subset of genes (1) and computing their mean fractional rank (MFR-subset(1) or MFR-positive),
i) isolating from the fractional ranks obtained at step g) the fractional rank obtained for the genes of the subset of genes (2) and computing their mean fractional rank (MFR-subset(2) or MFR-negative), and
e) computing a second deR score as MFR-subset(1) less MFR-subset(2), and
k) comparing said first deR score with said second deR score, wherein an observed deviation between said first and said second deR score may be indicative of a progression or a regression of said cancer.

In some embodiments, the second deR score may be greater than the first deR score, then the cancer may be observed as progressing, and when the second deR score may be lower than the first deR score, then the cancer may be observed as regressing.

In some embodiments the disclosure relates to a method for monitoring a progression or a regression a cancer in a subject presumed or known to have a TEAD-active cancer, the method comprising the use of a set of gene transcripts from a set of genes (B), and the method comprising at least the steps of:
a) obtaining, from a first biological sample obtained from said subject at a first time, a level of each gene transcript of said set of genes,
b) multiplying each level of each gene transcript of said set of genes by a coefficient associated with each gene, to obtain, for each gene, a product (Pgeneᵢ) wherein genes refers to a gene listed in said set of genes;
c) summing the products Pgeneᵢ (ΣPgeneᵢ) obtained at step b) and adding a constant for obtaining a first (S) score: (ΣPgeneᵢ+constant),
d) obtaining, from a second biological sample obtained from said subject at a second time, subsequent to the first time, a level of each gene transcript of said set of genes,
e) multiplying each level of each gene transcript of said set of genes by a coefficient associated with each gene, to obtain, for each gene, a product (Pgeneᵢ wherein gene, refers to a gene listed in said set of genes);
f) summing the products Pgeneᵢ (ΣPgeneᵢ) obtained at step e) and adding a constant for obtaining a second (S) score: (ΣPgeneᵢ+constant), and
g) comparing said first (S) score with said second (S) score, wherein an observed deviation between said first and said second (S) score may be indicative of a progression or a regression of said cancer,
wherein the coefficient(s) and the constant used at steps b), c), e), and f) may be previously obtained by a stepwise multiple linear regression analysis correlating (i) levels of said gene transcripts previously obtained in a third biological sample obtained from a subject having said cancer with (ii) levels of gene transcripts of a TEAD-500 signature obtained in a fourth biological sample from said subject, the fourth biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed in Table 2.

In some embodiments, the correlation operated with the stepwise multiple linear regression analysis may be carried between (i) the levels of said gene transcripts and (ii) a TEAD-score obtained with the levels of gene transcripts of a TEAD-500 signature.

The subject from whom are taken the third and fourth biological samples may be the same or may be different from the subject from whom are taken the first and second biological samples for computing the (S) scores. The third and fourth biological samples are representative of the cancer for which a method according to the disclosure is considered.

In some embodiments, the second (S) score may be greater than the first (S) score, then the cancer may be observed as progressing, and when the second (S) score may be lower than the first (S) score, then the cancer may be observed as regressing.

In some embodiments the disclosure relates to a method for diagnosing and treating a subject in need thereof, the method comprising the step of characterizing a TEAD activity status of a cancer of said subject, and the step of administering to said subject a TEAD-inhibitor treatment if the cancer is characterized as being TEAD-active,
wherein
for a method for characterizing a TEAD activity status of a cancer comprising the use of a set of gene transcripts from a set of genes (A), then the method comprises at least the steps of:
a) obtaining, from a biological sample obtained from said subject, a level of each gene transcript of said set of genes,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a deR score as MFR-subset(1) less MFR-subset(2),
f) comparing said computed deR score obtained at step e) with a deR score of reference, wherein an observed deviation between said computed deR score and said deR score of reference may be an indicative of a TEAD-active cancer, and
g) administering to said subject a TEAD-inhibitor treatment if the cancer is characterized as being TEAD-active.

In some embodiments the disclosure relates to a method for diagnosing and treating a subject in need thereof, the method comprising the step of characterizing a TEAD activity status of a cancer of said subject, and the step of administering to said subject a TEAD-inhibitor treatment if the cancer is characterized as being TEAD-active,
wherein
for a method for characterizing a TEAD activity status of a cancer comprising the use of a set of gene transcripts from a set of genes (B), then the method comprises at least the steps of:
a) obtaining, from a biological sample obtained from said subject, a level of each gene transcript of said set of genes,
b) multiplying each obtained level of each gene transcript of said set of genes by a coefficient associated with each gene, to obtain, for each gene, a product (Pgeneᵢ) wherein gene; refers to a gene listed in said set of genes;
c) summing the products Pgeneᵢ (ΣPgeneᵢ) obtained at step b) and adding a constant for obtaining a (S) score: (ΣPgeneᵢ+constant),
d) comparing said computed (S) score obtained at step e) with a (S) score of reference, wherein an observed deviation between said computed (S) score and said (S) score of reference may be an indicative of a TEAD-active cancer, and
e) administering to said subject a TEAD-inhibitor treatment if the cancer is characterized as being TEAD-active,
wherein the coefficient(s) and the constant used at steps b) and c) may be previously obtained by a stepwise multiple linear regression analysis correlating (i) levels of said gene transcripts previously obtained in a first biological sample obtained from a subject having said cancer with (ii) levels of gene transcripts of a TEAD-500 signature obtained in a second biological sample from said subject, the second biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed in Table 2.

In some embodiments, the correlation operated with the stepwise multiple linear regression analysis may be carried between (i) the levels of said gene transcripts and (ii) a TEAD-score obtained with the levels of gene transcripts of a TEAD-500 signature.

The subject from whom are taken the first and second biological samples may be the same or may be different from the subject from whom is taken a biological sample for computing a (S) score. The first and second biological samples are representative of the cancer for which a method according to the disclosure is considered.

In some embodiments, the deR or (S) score may be greater than the value of reference, for example of about 0.055 for a (S) score, then the cancer may be characterized as being TEAD-active and a TEAD-inhibitor treatment is administered to said subject.

In some embodiments, the deR or (S) score may be less than or equal to the value of reference, for example of about 0.055 for a (S) score, then the cancer may be characterized as being TEAD-inactive and a TEAD-inhibitor treatment is not administered to said subject.

In some embodiments the disclosure relates to a method for predicting a response of a subject presumed to or known to have a TEAD-active cancer to a TEAD-inhibitor treatment,
the method comprising the use of a set of gene transcripts from a set of genes (A), and the method comprising at least the steps of:
a) obtaining, from a biological sample obtained from said subject, a level of each gene transcript of said set of genes,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a deR score as MFR-subset(1) less MFR-subset(2),
d) comparing said computed deR score obtained at step e) with a deR score of reference, wherein an observed deviation between said computed deR score and said deR score of reference may be predictive of a responsive or non-responsive subject to a TEAD-inhibitor treatment.

In some embodiments the disclosure relates to a method for predicting a response of a subject presumed to or known to have a TEAD-active cancer to a TEAD-inhibitor treatment,
the method comprising the use of a set of gene transcripts from a set of genes (B), and the method comprising at least the steps of:
a) obtaining, from a biological sample obtained from said subject, a level of each gene transcript of said set of genes,
b) multiplying each obtained level of each gene transcript of said set of genes by a coefficient associated with each gene, to obtain, for each gene, a product (Pgeneᵢ wherein gene; refers to a gene listed in said set of genes);
c) summing the products Pgeneᵢ (ΣPgeneᵢ) obtained at step b) and adding a constant for obtaining a (S) score: (ΣPgeneᵢ+constant),
d) comparing said computed (S) score obtained at step e) with a (S) score of reference, wherein an observed deviation between said computed (S) score and said (S) score of reference may be predictive of a responsive or non-responsive subject to a TEAD-inhibitor treatment,
wherein the coefficient(s) and the constant used at steps b) and c) may be previously obtained by a stepwise multiple linear regression analysis correlating (i) levels of said gene transcripts previously obtained in a first biological sample obtained from a subject having said cancer with (ii) levels of gene transcripts of a TEAD-500 signature obtained in a second biological sample from said subject, the second biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed in Table 2.

The subject from whom are taken the first and second biological samples may be the same or may be different from the subject from whom is taken a biological sample for computing a (S) score. The first and second biological samples are representative of the cancer for which a method according to the disclosure is considered.

In some embodiments, the deR or (S) score may be greater than the value of reference, for example of about 0.055 for a (S) score, then the cancer may be predicted as being responsive to a TEAD-inhibitor treatment, and when the deR or (S) score may be less than or equal to the value of reference, for example of about 0.055 for a (S) score, then the cancer may be characterized as being non-responsive to a TEAD-inhibitor treatment.

In some embodiments the disclosure relates to a method for monitoring a response of a subject presumed to or known to have a TEAD-active cancer to a TEAD-inhibitor treatment, the method comprising the use of a set of gene transcripts from a set of genes (A), and the method comprising at least the steps of:
a) obtaining, from a first biological sample obtained from said subject before administration of a TEAD-inhibitor treatment, a level of each gene transcript of said set of genes,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a first deR score as MFR-subset(1) less MFR-subset(2),
f) obtaining, from a second biological sample obtained from said subject after administration of a TEAD-inhibitor treatment, a level of each gene transcript of said set of genes,
g) for each gene transcript of said set of genes, converting the level of each gene transcript obtained at step f) into a fractional rank by dividing the rank of the gene having said level of said gene transcript by the number of genes from said set of genes,
h) isolating from the fractional ranks obtained at step g) the fractional rank obtained for the genes of the subset of genes (1) and computing their mean fractional rank (MFR-subset(1) or MFR-positive),
i) isolating from the fractional ranks obtained at step g) the fractional rank obtained for the genes of the subset of genes (2) and computing their mean fractional rank (MFR-subset(2) or MFR-negative), and
e) computing a second deR score as MFR-subset(1) less MFR-subset(2), and
k) comparing said first deR score with said second deR score, wherein an observed deviation between said first and said second deR score may be indicative of an effective or ineffective TEAD-inhibitor treatment.

In some embodiments, when the second deR score is greater than the first deR score, then the TEAD-inhibitor treatment may be observed as being ineffective against the cancer, and when the second deR score is lower than the first deR score, then the TEAD-inhibitor treatment may be observed as being effective against the cancer.

In some embodiments the disclosure relates to a method for monitoring a response of a subject presumed or known as having a TEAD-active cancer to a TEAD-inhibitor treatment, the method comprising the use of a set of gene transcripts from a set of genes (B), and the method comprising at least the steps of:
a) obtaining, from a first biological sample obtained from said subject before administration of a TEAD-inhibitor treatment, a level of each gene transcript of said set of genes,
b) multiplying each obtained level of each gene transcript of said set of genes by a coefficient associated with each gene, to obtain, for each gene, a product (Pgeneᵢ) wherein gene; refers to a gene listed in said set of genes;
c) summing the products Pgeneᵢ (ΣPgeneᵢ) obtained at step b) and adding a constant for obtaining a first (S) score: (ΣPgeneᵢ+constant),
d) obtaining, from a second biological sample obtained from said subject after administration of a TEAD-inhibitor treatment, a level of each gene transcript of said set of genes,
e) multiplying each obtained level of each gene transcript of said set of genes by a coefficient associated with each gene, to obtain, for each gene, a product (Pgeneᵢ wherein gene; refers to a gene listed in said set of genes);
f) summing the products Pgeneᵢ (ΣPgeneᵢ) obtained at step e) and adding a constant for obtaining a second (S) score: (ΣPgeneᵢ+constant), and
g) comparing said first (S) score with said second (S) score, wherein an observed deviation between said first and said second (S) score may be indicative of an effective or ineffective TEAD-inhibitor treatment,
wherein the coefficient(s) and the constant used steps b), c), e) and f) may be previously obtained by stepwise multiple linear regression analysis for correlating (i) levels of said gene transcripts previously obtained in a third biological sample obtained from a subject having said cancer with (ii) levels of gene transcripts of a TEAD-500 signature obtained in a fourth biological sample obtained from said subject, the fourth biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed in Table 2.

In some embodiments, the correlation operated with the stepwise multiple linear regression analysis may be carried between (i) the levels of said gene transcripts and (ii) a TEAD-score obtained with the levels of gene transcripts of a TEAD-500 signature.

The subject from whom are taken the third and fourth biological samples may be the same or may be different from the subject from whom are taken the biological samples for computing the (S) scores. The third and fourth biological samples are representative of the cancer for which a method according to the disclosure is considered.

In some embodiments, when the second (S) score is greater than the first (S) score, then the TEAD-inhibitor treatment may be observed as being ineffective against the cancer, and when the second (S) score is lower than the first (S) score, then the TEAD-inhibitor treatment may be observed as being effective against the cancer.

In the methods above disclosed, the gene transcripts of the set of gene transcripts (A) or (B) may be obtained from a biological sample as above indicated.

In the methods above disclosed, the gene transcripts of the set of gene transcripts (A) or (B) may be obtained from a body fluid sample as above indicated.

In the methods above disclosed, the gene transcripts of the set of gene transcripts (A) or (B) may be obtained from EVs as above indicated.

In some embodiments the disclosure relates to a method for screening a TEAD-inhibitor candidate for inhibiting a TEAD activity, the method comprising the use of a set of gene transcripts from a set of genes (A), and the method comprising at least the steps of:
a) obtaining, from a first biological sample obtained from a supernatant of a cell culture, the first biological sample being obtained before contacting the cell culture with said TEAD-inhibitor candidate compound, a level of each gene transcript of said set of genes,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a first deR score as MFR-subset(1) less MFR-subset(2),
f) obtaining, from a second biological sample obtained from a supernatant of said cell culture, the second biological sample being obtained after contacting said cell culture with said TEAD-inhibitor candidate compound, a level of each gene transcript of said set of genes,
g) for each gene transcript of said set of genes, converting the level of each gene transcript obtained at step f) into a fractional rank by dividing the rank of the gene having said level of said gene transcript by the number of genes from said set of genes,
h) isolating from the fractional ranks obtained at step g) the fractional rank obtained for the genes of the subset of genes (1) and computing their mean fractional rank (MFR-subset(1) or MFR-positive),
i) isolating from the fractional ranks obtained at step g) the fractional rank obtained for the genes of the subset of genes (2) and computing their mean fractional rank (MFR-subset(2) or MFR-negative), and
e) computing a second deR score as MFR-subset(1) less MFR-subset(2), and
k) comparing said first deR score with said second deR score, wherein an observed deviation between said first and said second deR score may be indicative of an effective or ineffective TEAD-inhibitor candidate compound for inhibiting a TEAD activity.

In some embodiments, the second deR score may be greater than the first deR score, then the TEAD-inhibitor candidate compound may be observed as being ineffective for inhibiting a TEAD activity, and when the second deR score may be lower than the first deR score, then the TEAD-inhibitor candidate compound may be observed as being effective for inhibiting a TEAD activity.

In some embodiments the disclosure relates to a method for screening a TEAD-inhibitor candidate for inhibiting a TEAD activity, the method comprising the use of a set of gene transcripts from a set of genes (B), and the method comprising at least the steps of:
a) obtaining, from a first biological sample obtained from a supernatant of a cell culture, the first biological sample being obtained before contacting the cell culture with said TEAD-inhibitor candidate compound, a level of each gene transcript of said set of genes,
b) multiplying each obtained level of each gene transcript of said set of genes by a coefficient associated with each gene, to obtain, for each gene, a product (Pgeneᵢ) wherein gene; refers to a gene listed in said set of genes;
c) summing the products Pgeneᵢ (ΣPgeneᵢ) obtained at step b) and adding a constant for obtaining a first (S) score: (ΣPgeneᵢ+constant),
d) obtaining, from a second biological sample obtained from a supernatant of said cell culture, the second biological sample being obtained after contacting said cell culture with said TEAD-inhibitor candidate compound, a level of each gene transcript of said set of genes,
e) multiplying each obtained level of each gene transcript of said set of genes by a coefficient associated with each gene, to obtain, for each gene, a product (Pgeneᵢ) wherein gene; refers to a gene listed in said set of genes;
f) summing the products Pgeneᵢ (ΣPgeneᵢ) obtained at step e) and adding a constant for obtaining a second (S) score: (ΣPgeneᵢ+constant), and
g) comparing said first (S) score with said second (S) score, wherein an observed deviation between said first and said second (S) score may be indicative of an effective or ineffective TEAD-inhibitor treatment,
wherein the coefficient(s) and the constant used at steps b), c), e) and f) may be previously obtained by stepwise multiple linear regression analysis for correlating (i) levels of said gene transcripts previously obtained in a third biological sample obtained from a supernatant of cell culture with (ii) levels of gene transcripts of a TEAD-500 signature obtained in a fourth biological sample obtained from said cell culture, the fourth biological sample being from cancer cells from said subject, wherein the TEAD-500 signature comprises the gene transcripts of a set of genes comprising any of 220 to 249 of genes of a subset of genes (1) and any of 210 to 233 of genes of a subset of genes (2) as disclosed in Table 2.

In some embodiments, the correlation operated with the stepwise multiple linear regression analysis may be carried between (i) the levels of said gene transcripts and (ii) a TEAD-score obtained with the levels of gene transcripts of a TEAD-500 signature.

The cell culture from which are taken the third and fourth biological samples may be the same or may be different from the cell culture from which are taken the biological samples for computing the (S) scores.

A suitable cell for a cell culture in accordance with the disclosure may be a TEAD-active cell.

In some embodiments, the second (S) score may be greater than the first (S) score, then the TEAD-inhibitor candidate compound may be observed as being ineffective for inhibiting a TEAD activity, and when the second (S) score may be lower than the first (S) score, then the TEAD-inhibitor candidate compound may be observed as being effective for inhibiting a TEAD activity.

In some embodiments, a biological sample may be as above indicated.

In some embodiments, the set of gene transcripts may be obtained from extracellular vesicles.

The contacting of a cell culture with a TEAD-inhibitor candidate compound is carried out in conditions suitable for the TEAD-pathway to be inhibited by said compound.

In some embodiments the disclosure relates to a method for screening a TEAD-inhibitor candidate for inhibiting a TEAD activity, the method comprising the use of a set of gene transcripts from a set of genes (A), and the method comprising at least the steps of:
a) obtaining a level of each gene transcript of said set of genes, from a first biological sample obtained from an animal model of a TEAD-active cancer, said first biological sample being obtained before administering to said animal model of a TEAD-active cancer said TEAD-inhibitor candidate compound,
b) for each gene transcript of said set of genes, converting the level of each gene transcript obtained at step a) into a fractional rank by dividing the rank of the gene having said level of said gene transcript by the number of genes from said set of genes,
c) isolating from the fractional ranks obtained at step b) the fractional rank obtained for the genes of the subset of genes (1) and computing their mean fractional rank (MFR-subset(1) or MFR-positive),
d) isolating from the fractional ranks obtained at step c) the fractional rank obtained for the genes of the subset of genes (2) and computing their mean fractional rank (MFR-subset(2) or MFR-negative), and
e) computing a first deR score as MFR-subset(1) less MFR-subset(2),
f) obtaining a level of each gene transcript of said set of genes, from a second biological sample obtained from said animal model of a TEAD-active cancer, the second biological sample being obtained after administering to said animal model of a TEAD-active cancer said TEAD-inhibitor candidate compound,
g) for each gene transcript of said set of genes, converting the level of each gene transcript obtained at step f) into a fractional rank by dividing the rank of the gene having said level of said gene transcript by the number of genes from said set of genes,
h) isolating from the fractional ranks obtained at step g) the fractional rank obtained for the genes of the subset of genes (1) and computing their mean fractional rank (MFR-subset(1) or MFR-positive),
i) isolating from the fractional ranks obtained at step g) the fractional rank obtained for the genes of the subset of genes (2) and computing their mean fractional rank (MFR-subset(2) or MFR-negative), and
e) computing a second deR score as MFR-subset(1) less MFR-subset(2), and
k) comparing said first deR score with said second deR score, wherein an observed deviation between said first and said second deR score may be indicative of an effective or ineffective TEAD-inhibitor candidate compound for inhibiting a TEAD activity.

In some embodiments, the second deR score may be greater than the first deR score, then the TEAD-inhibitor candidate compound may be observed as being ineffective for inhibiting a TEAD activity, and when the second deR score may be lower than the first deR score, then the TEAD-inhibitor candidate compound may be observed as being effective for inhibiting a TEAD activity.

In some embodiments the disclosure relates to a method for screening a TEAD-inhibitor candidate for inhibiting a TEAD activity, the method comprising the use of a set of gene transcripts from a set of genes (B), and the method comprising at least the steps of:
a) obtaining a level of each gene transcript of said set of genes, from a first biological sample obtained from an animal model of a TEAD-active cancer, the first biological sample being obtained before administering to said animal model of a TEAD-active cancer said TEAD-inhibitor candidate compound,
b) multiplying each obtained level of each gene transcript of said set of genes by a coefficient associated with each gene, to obtain, for each gene, a product (Pgeneᵢ) wherein gene; refers to a gene listed in said set of genes;
c) summing the products Pgeneᵢ (ΣPgeneᵢ) obtained at step b) and adding a constant for obtaining a first (S) score: (ΣPgeneᵢ+constant),
d) obtaining a level of each gene transcript of said set of genes, from a second biological sample obtained from said animal model of a TEAD-active cancer, the second biological sample being obtained after administering to said animal model of a TEAD-active cancer said TEAD-inhibitor candidate compound,
e) multiplying each obtained level of each gene transcript of said set of genes by a coefficient associated with each gene, to obtain, for each gene, a product (Pgeneᵢ) wherein gene; refers to a gene listed in said set of genes;
f) summing the products Pgeneᵢ (ΣPgeneᵢ) obtained at step e) and adding a constant for obtaining a second (S) score: (ΣPgeneᵢ+constant), and
g) comparing said first (S) score with said second (S) score, wherein an observed deviation between said first and said second (S) score may be indicative of an effective or ineffective TEAD-inhibitor treatment,
wherein the coefficient(s) and the constant used at steps b), c), e) and f) may be previously obtained by stepwise multiple linear regression analysis for correlating (i) levels of said gene transcripts previously obtained in a third biological sample obtained from an animal model of a TEAD-active cancer with (ii) levels of gene transcripts of a TEAD-500 signature obtained in a fourth biological sample obtained from said animal model, the fourth biological sample being from cancer cells from said animal model, wherein the TEAD-500 signature comprises the gene transcripts of a set of genes comprising any of 220 to 249 of genes of a subset of genes (1) and any of 210 to 233 of genes of a subset of genes (2) as disclosed in Table 2.

In some embodiments, the correlation operated with the stepwise multiple linear regression analysis may be carried between (i) the levels of said gene transcripts and (ii) a TEAD-score obtained with the levels of gene transcripts of a TEAD-500 signature.

The animal model from whom are taken the third and fourth biological samples may be the same or may be different from the animal model from which are taken the biological samples for computing the (S) scores.

The third and fourth biological samples are representative of the cancer for which a method according to the disclosure is considered.

In some embodiments, the second (S) score may be greater than the first (S) score, then the TEAD-inhibitor candidate compound may be observed as being ineffective for inhibiting a TEAD activity, and when the second (S) score may be lower than the first (S) score, then the TEAD-inhibitor candidate compound may be observed as being effective for inhibiting a TEAD activity.

In some embodiments, the first, second and third biological samples may be as above indicated.

In some embodiments, the set of gene transcripts may be obtained from extracellular vesicles.

In some embodiments, the correlation operated with the stepwise multiple linear regression analysis may be carried between (i) the levels of said gene transcripts and (ii) a TEAD-score obtained with the levels of gene transcripts of a TEAD-500 signature.

### Cancers

In some embodiments, the cancer may be selected among mesothelioma, adrenocortical carcinoma, bladder urothelial carcinoma, breast invasive carcinoma, cervical squamous cell carcinoma and endocervical adenocarcinoma, cholangiocarcinoma, colorectal cancer, consensus molecular subtypes 1 of colorectal cancer, consensus molecular subtypes 2 of colorectal cancer, consensus molecular subtypes 3 of colorectal cancer, consensus molecular subtypes 4 of colorectal cancer, colon adenocarcinoma, lymphoid neoplasm diffuse large b-cell lymphoma, esophageal carcinoma, glioblastoma multiforme, head and neck squamous cell carcinoma, kidney renal clear cell carcinoma, kidney renal papillary cell carcinoma, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, and uterine carcinosarcoma.

In some embodiments, the cancer may be a mesothelioma.

In some embodiments, the cancer may be a colorectal cancer.

### Kits and Articles of Manufacture

In some embodiments, provided herein is a kit or an article of manufacture containing probes, such as nucleic acids, for detecting and quantifying any of the gene transcripts of a set of genes disclosed herein.

In some embodiments, the disclosure relates to a kit comprising a solid support comprising a panel of nucleic acid for obtaining a level of gene transcripts of a set of genes as disclosed herein.

A kit of the disclosure is suitable for use in the uses and methods disclosed herein.

In some embodiments, the kit or article of manufacture may include one or more reagents for preparing a biological sample, or a fraction thereof, for RNA sequencing analysis.

In some embodiments, the kit or article of manufacture further includes one or more reagents for determining the levels of the gene transcripts disclosed herein from a biological sample.

In some embodiments, the kit or article of manufacture may include instructions to use the kit for any of the methods/uses as described herein.

In some embodiments, the kit or article of manufacture may include a container, a label on the container, and a composition contained within the container, wherein the composition includes one or more polynucleotides that hybridize to a gene transcript listed herein under stringent conditions, and the label on the container indicates that the composition can be used to obtain the levels of the genes transcripts listed herein, and wherein the kit includes instructions for using the polynucleotide(s) for evaluating the presence and quantifying the levels of the gene transcripts of the disclosure in a biological sample, or a fraction thereof.

In some embodiments, the kit or article of manufacture is oligonucleotide-based and may include, for example (1) an oligonucleotide, for example a detectably labeled oligonucleotide, which hybridizes to a gene transcript or (2) a pair of primers useful for amplifying a gene transcript. In some embodiments, the kit or article of manufacture may also include a buffering agent, a preservative, or a stabilizing agent. In some embodiments, the kit or article of manufacture may further include components necessary for detecting the detectable label, for example, an enzyme or a substrate. In some embodiments, the kit or article of manufacture may also contain a control sample or a series of control samples that can be assayed and compared to the test sample. In some embodiments, each component of the kit or article of manufacture can be enclosed within an individual container and all of the various containers can be within a single package, along with instructions for interpreting the results of the assays performed using the kit or article of manufacture.

### TEAD-Pathway Inhibitors

In some embodiments, a TEAD-pathway inhibitor (or TEAD-inhibitor) is administered to a subject in need thereof. A subject in need thereof may be a subject identified as having a TEAD-active cancer (or known as having a TEAD-active cancer), or as likely to respond to a TEAD-pathway inhibitor (or presumed as having a TEAD-active cancer) using the methods and set of gene transcripts described herein.

Any TEAD-pathway inhibitor known in the art may be administered.

As used herein, a “TEAD-pathway inhibitor” may be a small or large molecule that inhibits the HIPPO-YAP/WWTR1/TEAD pathway.

As example of suitable TEAD-inhibitors, one may cite:

Statins: Statins have been reported to inhibit TEAD activity by reducing the expression of YAP.

Epigallocatechin gallate (EGCG): EGCG has been shown to inhibit TEAD activity by disrupting the interaction between TEAD and its co-activator YAP.

Cucurbitacin B: Cucurbitacin B has been shown to inhibit TEAD activity by disrupting the interaction between TEAD and its co-activator YAP.

CA3: CA3 is a synthetic compound that has been shown to inhibit TEAD activity by disrupting the interaction between TEAD and its co-activator YAP.

CA-170: CA-170 is a small molecule inhibitor of both PD-L1 and VISTA (V-domain Ig suppressor of T cell activation) that has been shown to have TEAD-inhibiting activity. A Phase I clinical trial of CA-170 in patients with advanced solid tumors is ongoing.

YAPi: YAPi is a monoclonal antibody that targets the YAP protein, which is a co-activator of TEAD. A Phase I clinical trial of YAPi in patients with advanced solid tumors is ongoing.

CYT-0851: CYT-0851 is a small molecule inhibitor of the bromodomain and extraterminal domain (BET) proteins that has been shown to have TEAD-inhibiting activity. A Phase I clinical trial of CYT-0851 in patients with advanced solid tumors is ongoing.

CB-839: CB-839 is a small molecule inhibitor of glutaminase that has been shown to inhibit TEAD activity in preclinical studies. A Phase I/II clinical trial of CB-839 in combination with a PD-L1 inhibitor in patients with advanced solid tumors is ongoing.

Suitable compounds for the disclosure may be 1H-indolyl-acrylamide derivatives of formula (1) disclosed in WO 2021/204823:

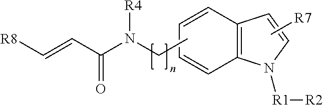

wherein
   n is an integer chosen from 0 and 1
   R1 is chosen from a single bond, and a (C1-C4) alkenyl group,
   R2 is chosen from: a (C1-C4) alkyl group or substituted with one or more fluorine atoms, a (C1-C3) alkoxy group substituted with one or more fluorine atoms, a phenyl group unsubstituted or substituted with one or more R3 groups, a (C4-C8) cycloalkyl group unsubstituted or substituted with one or more R5 groups, a (C4-C8) heterocyclyl group unsubstituted or substituted with one or more R6 groups, and a NR9R10 group,
   R3 is chosen from a (C1-C4) alkyl group unsubstituted or substituted with one or more fluorine atoms, a cyclopropyl group, a halogen atom, a (C1-C3) alkoxy group unsubstituted or substituted with one or more fluorine atoms, a pentafluorosulfanyl group, a nitrile group, a (C1-C3) trialkylsilyl group, a (C1-C3) alkylsulfonyl group, and a phenyl group unsubstituted or substituted with a trifluoromethyl group,
   R4 is chosen from a hydrogen atom and a (C1-C4) alkyl group,
   R5 is chosen from a fluorine atom and a trifluoromethyl group,
   R6 is chosen from a phenyl group unsubstituted or substituted with one or more fluorine atoms or one or more CF3 groups, a (C1-C4) alkyl group substituted with one or more fluorine atoms, and a fluorine atom,
   R7 is chosen from a hydrogen atom, a nitrile group, and a (C1-C4) alkyl group,
   R8 is chosen from a hydrogen atom and a (C1-C4) alkyl group unsubstituted or substituted with a di(C1-C4) alkylamino group,
   R9 and R10 are identical or different and chosen from an (C1-C3) alkyl group unsubstituted or substituted with one or more fluorine atoms,
   or a pharmaceutically acceptable salt thereof.

As suitable 1H-indolyl-acrylamide derivatives of formula (1), one may cite N-(1-(3-(trifluoromethyl)benzyl-1H-indol-5-yl)acrylamide.

### TEAD-500 Signature

A transcriptional signature (“TEAD-signature” or “TEAD-500 signature”) suitable for the disclosure may be obtained by measure of the levels of gene transcripts of a set of genes comprising any of 220 to 249 of genes of a subset of genes (1) (the positive effector genes) and any of 210 to 233 of genes of a subset of genes (2) (the negative effector genes) as set out in Table 2.

TABLE 2
Subset of genes (1) - Positive genes effector
AASS, ABAT, ACAT2, ADAMTS1, ADM, ADRB2, AMOT, ANXA3, ARHGAP11A, ARHGDIB,
AURKB, AVPI1, AXL, AZIN1, B4GALT4, BCAT1, BIRC5, BTG3, C4BPB, CAP2, CAV1, CAVIN1,
CCBE1, CCDC80, CCN1, CCN2, CDC25A, CDC6, CDCA3, CDCA4, CDCA5, CDCA8, CDH4,
CDK2, CDK6, CDV3, CENPA, CENPI, CENPM, CENPN, CHRNB1, CHST13, CKS2, CLDN1,
CLIC3, CNN3, COBL, COL8A1, COTL1, CPA4, CRIM1, CRY1, CTH, CXCL1, CYTH3, DAPK1,
DCLRE1B, DDAH1, DHCR7, DHFR, DIAPH3, DKK1, DLL1, DONSON, DUSP14, DUT, EBP,
EIF2AK3, EMG1, EPHA2, EPS8L2, ESM1, ETS1, EXO1, EXOSC2, F3, FAHD2A, FAM83D,
FANCA, FAT4, FDPS, FEN1, FMR1, FST, FSTL1, FSTL3, GADD45A, GADD45B, GINS1, GPC6,
GPR176, GPRC5A, GPRC5B, GRAMD2B, HASPIN, HEG1, HEXB, HPS5, HSPB11, IDI1, IGFBP7,
IKBIP, IL6, ITGB2, JDP2, JPH2, KPNA2, KRT8, KRT80, LCA5, LHFPL6, LMCD1, LMNB2, LRP8,
LRRFIP2, LSM5, LYPD6, LYRM1, MAD2L1, MAP6D1, MATN2, MATN3, MCM10, MCM2, MCM5,
MDC1, METRNL, MICB, MID1, MRPL33, MSRB3, MVD, MXRA7, NCAPD3, NEDD4, NEDD4L,
NEK2, NEXN, NFIB, NNMT, NOC3L, NTN4, NUAK1, NUAK2, NUDCD1, NUP107, NUP37,
OGFRL1, OLFML3, OLR1, OXCT1, PAK2, PCBD1, PCNA, PDLIM2, PDZD2, PEPD, PHLPP1,
PKMYT1, PKP2, PKP4, PLCE1, PLEKHA7, PLK2, PLOD2, PPIH, PRPS1, PRPS2, PRSS23,
PSG2, PSG6, PSG7, PSG9, PVR, PXMP2, QDPR, QKI, RAB11FIP1, RAB32, RACGAP1, RBM24,
RBMS2, RCN2, RFC4, RND3, RNF144B, ROR1, RPS24, SCD5, SCML1, SDC2, SEC14L1, SGK1,
SGMS2, SGTB, SH3RF1, SHCBP1, SKP2, SLC25A23, SLC25A3, SLC38A5, SLC3A2, SLC7A1,
SLC7A5, SMPD4, SNAPC1, SNX24, SORT1, SPAG1, SPATA5, STK3, STX11, STXBP6, SUSD2,
SUV39H1, SYDE2, TACC3, TAGLN, TEAD1, TEAD4, TENT5B, TGM2, THBS1, TK1, TMEM139,
TMEM160, TNFAIP3, TNFRSF12A, TNNC1, TPM1, TPX2, TRIP13, TSPAN2, TTF2, TUBB6,
TUFT1, TYMS, UAP1, UBE2C, UGCG, UHRF1, VKORC1L1, WWC1, WWC2, YAP1, ZBED2,
ZDHHC18, ZNF488, and ZNF704.
Subset of genes (2) - Negative genes effector
AASDH, ABCA1, ABCC5, ABI3BP, ABLIM3, ACADVL, ACOT11, ACOX2, ACSL5, ADAM28, AGL,
AGPAT4, ALDH3A2, ANKRD12, ANKRD22, ANKRD29, ANKRD42, ANTXR2, APBB3, ARAP3,
ARHGEF2, ASF1A, ATP7A, ATXN1, BCL11B, BHLHE41, BMF, CA2, CASP1, CBR3, CCNG2,
CDC42EP4, CDK1, CEBPB, CELSR3, CLCN3, CLDN4, COL6A1, COL6A2, CPE, CRABP2, CROT,
CSRNP2, CSTA, CTNNBIP1, CTSB, CTSK, CXXC5, CYP1B1, CYP27C1, DDR1, DEDD2, DHX32,
DIAPH2, DSC2, DSG3, DUSP6, DYNC2LI1, ELN, EPS8L3, ERAP2, FAM102A, FAM117B,
FAM83B, FAM89B, FERMT1, FKBP2, FOS, FTH1, FXYD3, GDPD1, GOLGA5, GOLPH3L,
GPNMB, GPRC5C, GRB10, GSN, HAS3, HBP1, HDAC1, HDHD2, HEY1, HOXA5, IFI44, IGSF3,
IGSF9, INTS3, IRAK2, IRF9, IRX5, ITGA2, KCNMA1, KCNMB3, KCNN4, KIFAP3, KLF10, KLF13,
KLHL3, KLK11, KRCC1, KRIT1, KRTDAP, LMTK3, LRP10, LTBP4, LXN, LYPD3, MALL, MANSC1,
MAPK13, MARCKSL1, MFSD1, MFSD5, MGST2, MGST3, MLLT11, MLPH, MMP13, MSX2,
MTMR11, MTMR9, MTSS1, MYO1A, NAGK, NAPEPLD, NCOA3, NFIL3, NPAS2, NRIP1, OAS1,
OAS2, OASL, OFD1, OSBPL7, OTUB2, OVOL1, PAG1, PAK1, PCDHB2, PCDHB9, PCGF3,
PCMTD2, PERP, PHF21A, PIK3C2B, PIK3R1, PIK3R2, PIK3R3, PIP4P2, PJA2, PKIA, PLA2G4C,
PNRC1, PPP1R11, PRRX2, PTPRE, PYGB, RAC2, RALGPS1, RAPGEFL1, RBM23, RBM45,
RBM47, RBP1, REEP6, RGL2, RGS17, RHOC, S100A14, SAMD9, SEC14L2, SECISBP2,
SH3PXD2A, SH3TC1, SHROOM2, SLC14A1, SLC1A2, SLC30A9, SLC35C1, SLC37A2,
SLC39A11, SLFN5, SLITRK6, SLK, SMOC1, SNCG, SP1, SPIRE2, SPRY4, SQSTM1, SRD5A3,
SSPN, STMN3, STX1A, TBX3, TCF25, TDO2, TET2, TFF1, TLR3, TMC4, TMC7, TMEM140,
TMEM144, TMEM45B, TP53INP1, TP63, TPD52L1, TRAPPC6B, TRIB1, TRIB2, TRIM13, TRIM31,
TRIM38, TRIOBP, TRIP11, TSC22D1, TSPAN1, TTC17, TTLL3, TUBB3, UBC, ULK1, VAMP8,
VGF, VPS52, VSNL1, WDR13, ZCWPW1, ZNF292, ZNF467, ZNF75D, and ZSWIM7.

The levels of the gene transcripts of a TEAD-signature may be measured as above described for the levels of the gene transcripts of the set of gene transcripts of the disclosure.

The levels of gene transcripts of a TEAD-signature are used herein in a stepwise multiple linear regression analysis for correlating (i) levels of the gene transcripts of the set of gene transcripts from a set of genes (B) with (ii) levels of the gene transcripts of a TEAD-500 signature.

A transcriptional signature (“TEAD signature”) may be obtained by measure of the levels of the gene transcripts of a set of genes comprising any of 430 to 482 genes above indicated, or any of the 435 to 482, or any of 440 to 482, or any of 445 to 482, or any of 450 to 482, or any of 455 to 482, or any of 460 to 482, or any of 465 to 482, or any of 470 to 482, or any of 475 to 482, or any of 480 to 482 genes above indicated.

A transcriptional signature (“TEAD signature”) may be obtained by measure of the levels of the gene transcripts of a set of genes comprising any of 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481 or 482 genes above indicated.

A transcriptional signature (“TEAD-signature”) according to the disclosure may be used to determine a TEAD score.

In some embodiments, a TEAD score may be used in a stepwise multiple linear regression analysis for correlating (i) the levels of the gene transcripts of a set of gene transcripts (B) with (ii) the TEAD score computed with the levels of the gene transcripts of a TEAD-500 signature.

In some embodiments, a TEAD score is calculated according to the following method:
a) measuring, in a biological sample, the level of each gene of said set of genes,
b) for each gene of said set of genes, converting the gene's expression level obtained at step a) into a fractional rank by dividing the rank of said gene by the number of genes from said set of genes,
c) isolating from the fractional ranks obtained at step b) the fractional rank obtained for the genes of the subset of genes (1) and computing their mean fractional rank (MFR-positive),
d) isolating from the fractional ranks obtained at step b) the fractional rank obtained for the genes of the subset of genes (2) and computing their mean fractional rank (MFR-negative), and
e) computing a deR score as MFR-positive-MFR-negative.

The ranking of a gene within a set of the gene is obtained by attributing to the gene having the lowest level of gene transcript the rank 1, and to the gene having the next highest level of gene transcript the rank 2, and so on until the gene with the highest level of gene transcript is given the highest rank. Genes having gene transcripts with equal levels are given the average rank, e.g., two genes with identical levels of gene transcripts with 0 expression are given the rank 1.5.

When the deR score is greater than about 0.055, then the cancer is TEAD-active, and when the deR score is less than or equal to about 0.055, then the cancer is TEAD-inactive.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. Thus, while the present invention has been described above with particularity, the following examples provide further detail in connection with what are presently deemed to be the most practical and preferred embodiments of the invention.

### Example 1: Materials & Methods

### Cells and Culture Conditions

All cell lines were grown at 37° C. under 5% CO₂. NCI-H226 (H226) (#CRL-5826), and HCT116 (#CCL-247) was purchased from the American Type Culture Collection (ATCC, Manassas, VA, USA) and cultured according to supplier's recommendation. SPC212 (No. 11120717) and Mero-14 (No. 09100101) were purchased from the European Collection of Authenticated Cell Cultures (ECACC, Public Health England, Salisbury, UK) and cultured according to supplier's recommendation. A short tandem repeat assay authenticated all cell lines at the MICROSYNTH AG (Balgach, Switzerland). PCR using the VENOR®GEM kit (BIOVALLEY, Nanterre, France) excluded mycoplasma infection.

### Cells Treatment

Cells were treated with or without (control cells) a TEAD-inhibitor (TEADi) at four doses: 0.0 μM, 0.3 μM, 1.0 μM & 3.0 μM for 24h.

EVs and cell RNAs were isolated and profiled by RNA-seq as detailed thereafter.

### Extracellular Vesicles (EVs) Preparation and RNA Extraction

Extracellular vesicles (EVs) were isolated from supernatants of treated and control cells using a membrane-based affinity column (EXOEASY, MAXI KIT, QIAGEN). Nanoparticle Tracking Analysis (NS300, MALVERN) was used to quantify and determine the Evs size distribution.

EVs RNAs were extracted using the EXORNEASY SERUM/PLASMA MAXI PLASMA KIT (QIAGEN).

Total cells RNA was extracted and purified using a MIRNEASY MINI KIT (QIAGEN, 217,004). The RNA concentration and purity were evaluated with an ND-1000 spectrophotometer (NANODROP, THERMO FISHER SCIENTIFIC, Wilmington, DE, USA). Quality metrics are the RNA Integrity Number (RIN) and DV200 value (% of RNA fragments with a length a ≥200 nt). 100 ng total RNA per sample was used as input material for the library preparation.

### RNA Sequencing

RNA-seq libraries were prepared using SMARTER® STRANDED TOTAL RNA-SEQ KIT V2—PICO INPUT MAMMALIAN for mRNA from TAKARA BIO USA, INC. Cell line mRNA sequencing was performed using KAPPA LIBRARY AMPLIFICATION KIT (ILLUMINA).

The libraries were sequenced on ILLUMINA NOVASEQ 6000.

The following steps were implemented for preparing the RNA-seq libraries.

### Step 1: DNAse Digestion

DNA depletion step using: DNAse I Amplification grade INVITROGEN #Cat. No. 18068-015 in the following conditions:
8 μl RNA/EVs samples, 1 μl of 10×DNAse I buffer, 1 μl of DNAse I, Amp Grade, 1 U/μl, then 15 minutes at room temperature.
DNAse inactivation by addition of 1 μl of 25 mM EDTA solution, then incubation at 65° C./10 minutes.
The EDTA was eliminated by using RNAESY MINELUTE (QIAGEN COLUMN)

Step 2: RNA seq preparation using TAKARA BIO USA, INC. SMARTER® STRANDED TOTAL RNA-SEQ KIT V2—PICO INPUT MAMMALIAN #634413. The vendor's (Takara) recommendation was followed with same specifications;
A) protocol: synthesis of cDNA
a. The option 2 was used (without fragmentation): starting from highly degraded RNA(EVs), then following TAKARA recommendation for the following steps:
B) protocol: adding ILLUMINA ADAPTERS/INDEX
C) protocol: purification of the RNA-seq library using AMPURE BEADS
D) protocol: ribosomal cDNA depletion with ZAPR V2 and R-PROBES V2
E) protocol: PCR 2—final amplification of the RNA-seq library
F) protocol: purification of final RNA-seq library using AMPURE BEADS

### TEAD-Activity Signatures

To estimate TEAD activity of the cells, a transcriptomic signature (TEAD-500 signature) from total cells RNA was used (Calvet, L., Dos-Santos, O., Spanakis, E. et al. 2022. YAP1 is essential for malignant mesothelioma tumor maintenance. BMC Cancer 22, 639. Doi.org/10.1186/s12885-022-09686 or PCT/EP2023/057332).YAP1/TEAD-dependent transcription was scored from RNA-seq data in single samples as the difference

deR=Rp−Rn

Rp is the mean fractional rank of the positive YAP1-effectors, and Rn, that of the negative effectors. The deR score is computed using only the levels of the genes constituting the signature.

A new signature comprising 28 gene transcripts was identified from the gene transcripts isolated from the Evs and used to compute a TEAD-activity score according to the method used for the “TEAD-500” signature, as above detailed or detailed in Calvet, L., Dos-Santos, O., Spanakis, E. et al. 2022. YAP1 is essential for malignant mesothelioma tumor maintenance. BMC Cancer 22, 639. Doi.org/10.1186/s12885-022-09686 or in PCT/EP2023/057332.

A stepwise multiple linear regression analysis was used for correlating the levels of all gene transcripts readily detected in the EVs (Log(FPKM+0.1) >4 at baseline) with the TEAD-500 signature score measured in the cells from which were obtained the EVs. This analysis resulted in a set of 8 genes (each with a specified coefficient) and a constant coefficient.

This second set of signatures having from 1 to 8 gene transcripts was identified from the gene transcripts isolated from the EVs. The second set of signatures was used to predict the TEAD-activity scores of parent tumor cells. TEAD-activity scores were calculated from the expression levels of downstream TEAD-effector genes highly correlated to TEAD modulation as previously described (Calvet, L., Dos-Santos, O., Spanakis, E. et al. 2022. YAP1 is essential for malignant mesothelioma tumor maintenance. BMC Cancer 22, 639. Doi.org/10.1186/s12885-022-09686 or in PCT/EP2023/057332).

For this second set of signatures comprising a set of gene transcripts from a set of genes, a predicted TEAD-activity score was computed as follows:
a) multiplication of each obtained level of each gene transcript of said set of genes by a coefficient associated with each gene, to obtain, for each gene, a product (Pgeneᵢ) wherein gene; refers to a gene listed in said set of genes;
b) summing the products Pgeneᵢ (ΣPgeneᵢ) obtained at step a) and adding a constant for obtaining a (S) score: (ΣPgeneᵢ+constant),
where the coefficient(s) and the constant used at steps a) and b) are previously obtained by a stepwise multiple linear regression analysis correlating (i) the levels of the EVs' gene transcripts with (ii) the score of a TEAD-500 signature measured in the cells from which the EVs were obtained.

The SPSS Statistics program (IBM; Armonk, NY) was used for stepwise multiple linear regression analysis to select a set of genes expressed in EVs and predicting the TEAD-500 value in parent cells. As well as for all other statistical work.

### Example 2: Results

The effects of TEAD inhibition on intracellular and EVs' RNAs isolated from the supernatant of three responding mesothelioma cells lines, NCI-H226, Mero-14, and SPC212, and one non-responding colon cell line, HCT116, were compared. Response was defined based on TEAD-inhibitor (TEADi) IC₅₀-assay (CELL TITER GLO®-PROMEGA). The cell lines, Mero14; Spc212, HCT116 and H226, were treated at four doses of TEADi (0.0 μM, 0.3 μM, 1.0 μM & 3.0 μM) for 24h. EVs and tumor RNAs were isolated and profiled by RNA-seq.

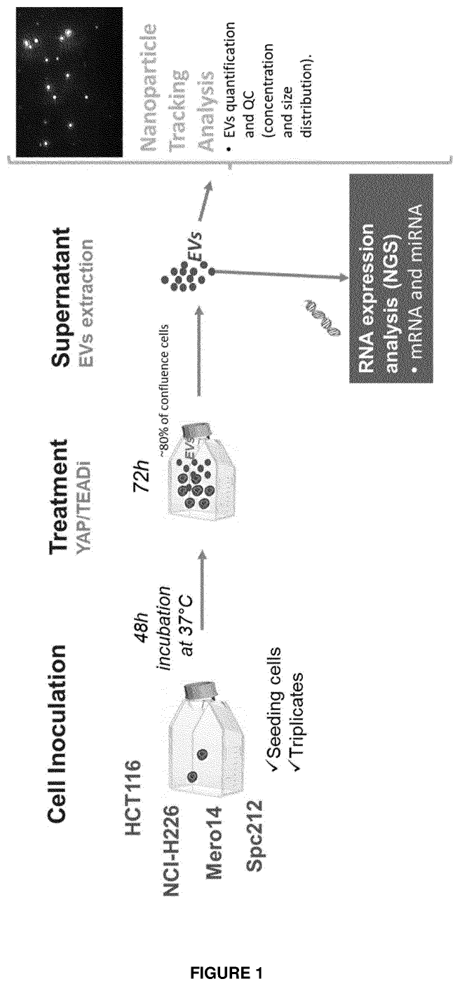

Process from cell treatment to Evs isolation and analysis is represented in FIG. 1.

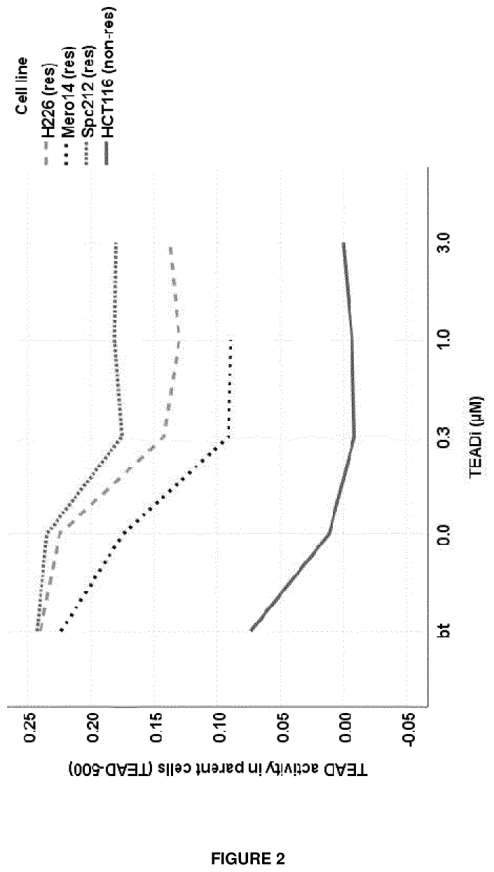

As shown in FIG. 2, the non-responder colon cell line HCT116 has lower intrinsic TEAD activity than the responding mesothelioma cell lines H226, Mero14 and SPC112. A significant drop is observed upon treatment in all cell lines, but the effect is more pronounced in the responders.

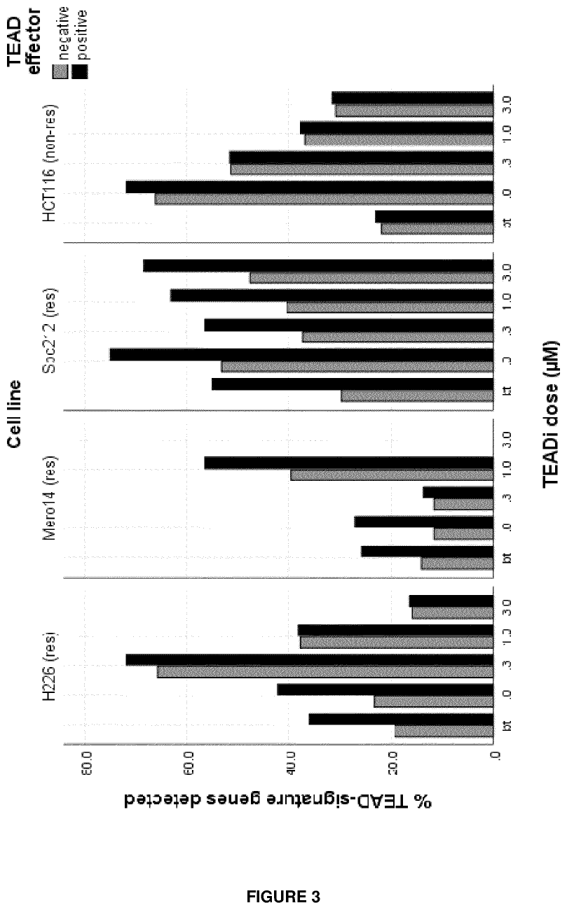

As shown in FIG. 3, about 50% of genes from the TEAD-500 signature were detected in the respective EVs' RNA across cell lines. Positive effectors designate genes whose expression level correlates positively with TEAD activity, and negative effectors designate genes whose expression correlates negatively.

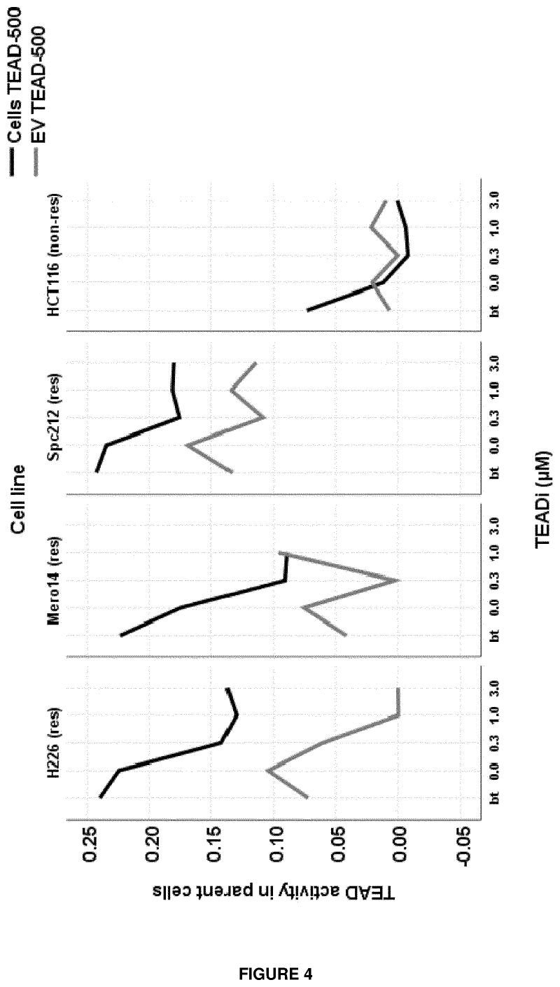

As shown in FIG. 4, using the published signature of ˜500 genes lower scores of TEAD activity from EVs' RNA were generally obtained compared to the scores of the parent cells. The correlation between the EVs and parent-cell values was poor (Mero14 r²=0.00; Spc212 r²=0.50, HCT116 r²=0.03) except for H226 (r²=0.66).

The TEAD-inhibitor caused a sharp drop of the TEAD activity score measured in EVs only for the H266 cell line but had no effect on the EVs values from the other two responders or the non-responder.

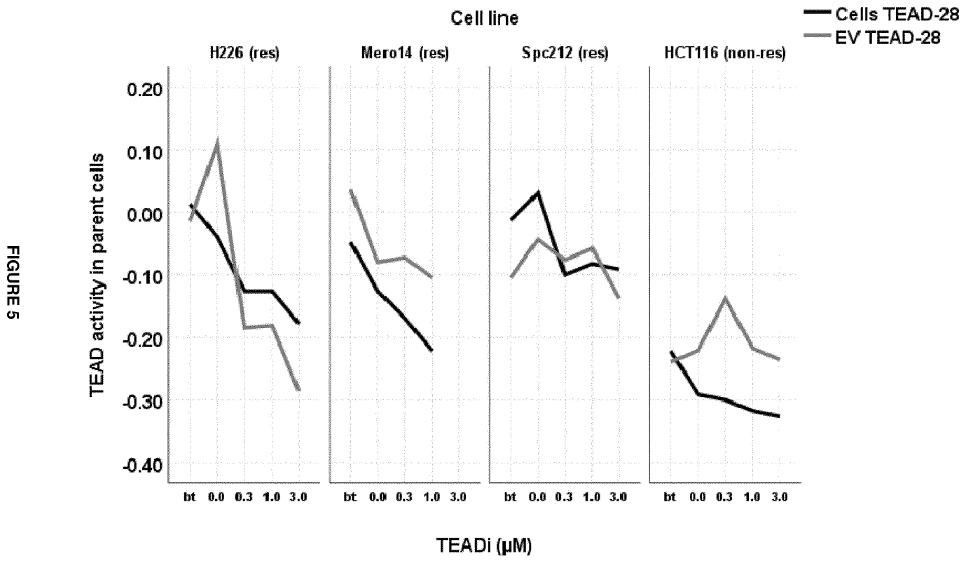

As shown in FIG. 5, focusing on a subset of 28 TEAD effectors (out of the ˜500) that were readily detected in EVs across the 3 mesothelioma cell lines, the correlation between the observed TEAD activity in parent cells and the score predicted from the EV RNA content was improved. TEADi-dose effects were observed in EVs' RNA from 2 of the 3 responding cell lines (H226 and Mero14). There was no correlation between EVs and parent-cell measurements in the negative control (HCT116).

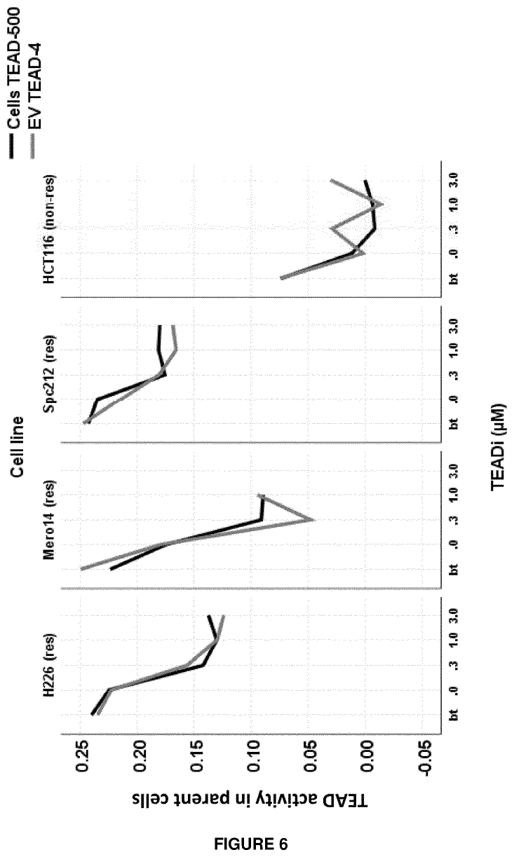

As shown in FIG. 6, a genome-wide, step-wise regression analysis to find a set of EVs' transcripts that successfully predict the TEAD-activity score of the parent cells was performed. Only transcripts that were readily detected at log 2FPKM >4 in the 3 responder cell lines at baseline were included in this analysis. A 4-transcript predictor was selected from this analysis. Using these 2 signatures, high correlations for all the responding cell lines (r2 for H226=0.96, Mero14=0.95, Spc212=0.89) but lower correlation (r2=0.63) for the non-responder cell line HCT116 were observed.

### Example 3: Conclusion

Despite their relatively lower yields compared to parent cells, EVs' RNAs are suitable for transcriptomic analysis.

More accurate estimation of TEAD activity can be achieved using a restricted effector-gene list of abundant EV transcripts. Novel biomarkers can be identified through genome-wide analysis of EVs' gene transcripts.

The EVs' gene transcripts derived signature which was identified for TEAD inhibition has the potential for use in plasma or other liquid biopsy as a peripheral marker for target engagement.

1. An isolated set of gene transcripts from a set of genes, said set of genes consisting of a subset of genes (1) consisting of ADM, AXL, BIRC5, CDV3, CRIM1, CTGF, CYR61, FSTL1, GADD45A, KRT8, LMNB2, MATN2, PKP4, RND3, RPS24, SEC14L1, SGK1, SLC25A3, SLC3A2, TNFRSF12A, TPM1, TPX2, and TUBB6 and of a subset of genes (2) consisting of CTSB, FTH1, SQSTM1, TCF25, and UBC.

2. An isolated set of gene transcripts consisting of at least two gene transcripts from a set of genes consisting of DLC1, AKAP2, CANX, SAFB2, EIF4H, NDUFS5, SEPT9, and EIF4A1.

3. The set of gene transcripts according to claim 2 consisting of a set of gene transcripts from a set of genes consisting of DLC1, AKAP2, CANX, and SAFB2, and optionally from at least one gene selected from EIF4H, NDUFS5, SEPT9, and EIF4A1.

4. The set of gene transcripts according to claim 1, where said set of gene transcripts is obtained from isolated extracellular vesicles.

5. Isolated extracellular vesicles comprising the set of gene transcripts as defined in claim 1 or comprising a set of gene transcripts consisting of at least one gene transcript from a set of genes consisting of DLC1, AKAP2, CANX, SAFB2, EIF4H, NDUFS5, SEPT9, and EIF4A1.

6. A method of determining a biomarker of a TEAD activity, for measuring or characterizing TEAD activity of a cancer or cell culture, for screening a TEAD-inhibitor candidate compound, or for diagnosing cancer, comprising using the set of gene transcripts according to claim 1 or a set of gene transcripts consisting of at least one gene transcript from a set of genes consisting of DLC1, AKAP2, CANX, SAFB2, EIF4H, NDUFS5, SEPT9, and EIF4A1, as a biomarker of a TEAD activity, for measuring or characterizing a TEAD activity of a cancer or of a cell culture, in a TEAD-inhibitor candidate compound screening method, or in a cancer diagnostic method.

7. A method for characterizing a TEAD activity status of a biological sample, for measuring a TEAD activity in a biological sample, for predicting a response of a cancer to a TEAD-inhibitor treatment in a subject known or presumed to have a TEAD-active cancer, for monitoring a response of a cancer to a TEAD-inhibitor treatment in a subject known or presumed to have a TEAD-active cancer, for predicting a cancer progression or regression in a subject known or presumed to have a TEAD-active cancer, for monitoring a cancer progression or regression in a subject known or presumed to have a TEAD-active cancer, or for screening a TEAD-inhibitor candidate compound, comprising using the set of gene transcripts according to claim 1 or a set of gene transcripts consisting of at least one gene transcript from a set of genes consisting of DLC1, AKAP2, CANX, SAFB2, EIF4H, NDUFS5, SEPT9, and EIF4A1, for characterizing a TEAD activity status of a biological sample, or for measuring a TEAD activity in a biological sample, or for predicting a response of a cancer to a TEAD-inhibitor treatment in a subject known or presumed to have a TEAD-active cancer, or for monitoring a response of a cancer to a TEAD-inhibitor treatment in a subject known or presumed to have a TEAD-active cancer, or for predicting a cancer progression or regression in a subject known or presumed to have a TEAD-active cancer, or for monitoring a cancer progression or regression in a subject known or presumed to have a TEAD-active cancer, or for screening a TEAD-inhibitor candidate compound.

8. The method according to claim 7, wherein said set of gene transcripts is obtained from isolated extracellular vesicles.

9. The method according to claim 7, wherein a level of each gene transcript is obtained.

10. The method according to claim 9, wherein a transcriptional signature is obtained from said gene transcript levels.

11. The method according to claim 10, wherein the obtained transcriptional signature is compared to a transcriptional signature of reference and wherein an observed deviation between said obtained transcriptional signature and said transcriptional signature of reference is indicative of a cancer being TEAD-active or TEAD-inactive, of a cancer susceptible to be responsive or not to the TEAD-inhibitor treatment, of an effective or ineffective TEAD-inhibitor treatment, of a TEAD-active cancer susceptible to progress or regress, of a progressing or regressing TEAD-active cancer, or of a TEAD-inhibitor candidate compound being effective or ineffective.

12. The method according to claim 9, wherein said gene transcript level is subject to a mathematical normalization.

13. The method according to claim 12:
wherein when a set of gene transcripts according to claim 1 is used, then the mathematical normalization computes a deR score according to a method comprising the steps of:
a) for each gene transcript of said set of genes, converting the level of each gene transcript into a fractional rank by dividing the rank of the gene having said level of said gene transcript by the number of genes from said set of genes,
b) isolating from the fractional ranks obtained at step a) the fractional rank obtained for the genes of the subset of genes (1) and computing their mean fractional rank (MFR-subset(1) or MFR-positive),
c) isolating from the fractional ranks obtained at step a) the fractional rank obtained for the genes of the subset of genes (2) and computing their mean fractional rank (MFR-subset(2) or MFR-negative), and
d) computing the deR score as MFR-subset(1) less MFR-subset(2)
or
wherein when a set of gene transcripts consisting of at least one gene transcript from a set of genes consisting of DLC1, AKAP2, CANX, SAFB2, EIF4H, NDUFS5, SEPT9, and EIF4A1 is used, then the mathematical normalization computes a (S) score according to a method comprising the steps of:
a) multiplying each level of each gene transcript of said set of genes by a coefficient associated with each gene, to obtain, for each gene, a product (Pgeneᵢ) wherein gene; refers to a gene listed in said set of genes;
b) summing the products Pgeneᵢ (SPgeneᵢ) obtained at step a) and adding a constant for obtaining a (S) score: (SPgeneᵢ+constant),
wherein the coefficient(s) and the constant used at steps a) and b) are previously obtained by a stepwise multiple linear regression analysis correlating (i) levels of said gene transcripts previously obtained in a first biological sample with (ii) levels of gene transcripts of a TEAD-500 signature previously measured in a second biological sample, wherein the TEAD-500 signature comprises the gene transcripts of a set of genes comprising any of 220 to 249 of genes of a subset of genes (1) and any of 210 to 233 of genes of a subset of genes (2) as disclosed in Table 2, the first and second biological samples being representative of a same TEAD-active cancer.

14. The method according to claim 13, wherein the reference value is a first deR or (S) score and the deR or (S) score to be compared to said value of reference is a second deR or (S) score subsequently measured to the first deR or (S) score.

15. The method according to claim 9, wherein said level of each gene transcript is obtained by RNA sequencing (RNA-seq) and is quantified as FPKM (Fragments Per Kilobase of transcript per Million mapped reads).

16. The method according to claim 7, wherein the cancer is selected among mesothelioma, adrenocortical carcinoma, bladder urothelial carcinoma, breast invasive carcinoma, cervical squamous cell carcinoma and endocervical adenocarcinoma, cholangiocarcinoma, colorectal cancer, consensus molecular subtypes 1 of colorectal cancer, consensus molecular subtypes 2 of colorectal cancer, consensus molecular subtypes 3 of colorectal cancer, consensus molecular subtypes 4 of colorectal cancer, colon adenocarcinoma, lymphoid neoplasm diffuse large b-cell lymphoma, esophageal carcinoma, glioblastoma multiforme, head and neck squamous cell carcinoma, kidney renal clear cell carcinoma, kidney renal papillary cell carcinoma, brain lower grade glioma, liver hepatocellular carcinoma, lung adenocarcinoma, lung squamous cell carcinoma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, and uterine carcinosarcoma.

17. A kit comprising a solid support comprising a panel of nucleic acid for obtaining gene transcript levels of a set of gene transcripts according to claim 1.
